SEQUENCE LISTING (10 sequences)

SEQ ID NO: 1 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 2 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 3 - protein, 890 aa, Homo sapiens
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 4 - protein, 20 aa, Artificial Sequence
  Synthetic polypeptide
LRMFSGTPVIRLRFKRLQPT

SEQ ID NO: 5 - protein, 11 aa, Artificial sequence
  Synthetic polypeptide
ELVGRVTSSGP

SEQ ID NO: 6 - protein, 8 aa, Artificial sequence
  Synthetic polypeptide
RNLVIKVN

SEQ ID NO: 7 - protein, 9 aa, Artificial sequence
  Synthetic polypeptide
DAVMKIAVA

SEQ ID NO: 8 - protein, 16 aa, Artificial sequence
  Synthetic polypeptide
ERGLYHLNLTVGIPFH

SEQ ID NO: 9 - protein, 17 aa, Artificial sequence
  Synthetic polypeptide
WLNGEDTTIQETVVNRM

SEQ ID NO: 10 - protein, 5 aa, Artificial sequence
  Synthetic polypeptide
GGGGS

(19) United States
(12) Patent Application Publication
(10) Pub. No.: US 2016/0266136 A1
(43) Pub. Date: Sep. 15, 2016

(54) HIGH-AFFINITY BINDING TO GAS6

(71) Applicant: The Board of Trustees of the Leland Stanford Junior University, Standford, CA (US)

(72) Inventors: Jennifer R. Cochran, Stanford, CA (US); Amato J. Giaccia, Stanford, CA (US); Yu Miao, Sunnyvale, CA (US); Mihalis Kariolis, Stanford, CA (US); Shiven Kapur, Stanford, CA (US); Irimpan I. Mathews, Menlo Park, CA (US)

(21) Appl. No.: 14/910,565

(22) PCT Filed: Dec. 12, 2013
(86) PCT No.: PCT/US13/74796

§ 371 Date: Feb. 5, 2016

Related U.S. Application Data
(60) Provisional application No. 61/872,602, filed on Aug. 30, 2013.

(51) Int. Cl. G01N 33/68

(52) U.S. Cl. CPC G01N 33/6827; G01N 2333/4706; G01N 2800/52; G01N 2800/7028; G01N 2500/20

(57) ABSTRACT

Compositions and methods are provided for measuring the amount of GAS6 in a sample. Also provided are compositions and methods related to high-affinity CAS6 inhibitor agents that induce a conformational change of GAS6 that stabilizes Helix A of GAS6.

### GOVERNMENT RIGHTS

This invention was made with Government support under contract DOE: DE-ACO3-76SF00515 awarded by the Department of Energy. The Government has certain rights in this invention.

### FIELD OF THE INVENTION

The present invention relates to polypeptides that exhibit high-affinity binding to Gas6 and an assay capable of measuring free Gas6 levels in a sample.

### BACKGROUND OF THE INVENTION

Invasion and metastasis are the most insidious and life-threatening aspects of cancer. While tumors with minimal or no invasion may be successfully removed, once the neoplasm becomes invasive, it can disseminate via the lymphatics and/or vascular channels to multiple sites, and complete removal becomes very difficult. Invasion and metastases kill hosts through two processes: local invasion and distant organ colonization and injury. Local invasion can compromise the function of involved tissues by local compression, local destruction, or prevention of normal organ function. The most significant turning point in cancer, however, is the establishment of distant metastasis. The patient can no longer be cured by local therapy alone at this point.

The process of metastasis is a cascade of linked sequential steps involving multiple host-tumor interactions. This complex process requires the cells to enter into the vascular or lymphatic circulation, arrest at a distant vascular or lymphatic bed, actively extravasate into the organ interstitium and parenchyma, and proliferate as a secondary colony. Metastatic potential is influenced by the local microenvironment, angiogenesis, stroma-tumor interactions, elaboration of cytokines by the local tissue, and by the molecular phenotype of the tumor and host cells.

Local microinvasion can occur early, even though distant dissemination may not be evident or may not yet have begun. Tumor cells penetrate the epithelial basement membrane and enter the underlying interstitial stroma during the transition from in situ to invasive carcinoma. Once the tumor cells invade the underlying stroma, they gain access to the lymphatics and blood vessels for distant dissemination while releasing matrix fragments and growth factors. General and widespread changes occur in the organization, distribution, and quantity of the epithelial basement membrane during the transition from benign to invasive carcinoma.

Therapeutic efforts in cancer prevention and treatment are being focused at the level of signaling pathways or selective modulatory proteins. Protein kinase activities, calcium homeostasis, and oncoprotein activation are driving signals and therefore may be key regulatory sites for therapeutic intervention. Kinases in signaling pathways regulating invasion and angiogenesis may be important regulators of metastasis. One of the largest classes of biochemical molecular targets is the family of receptor tyrosine kinases (RTKs). The most common receptor tyrosine kinase molecular targets to date are the EGF and vascular endothelial growth factor (VEGF) receptors. Newer kinase molecular targets include the type III RTK family of c-kit, and abl. Inhibitors of these molecules have been administered in combination with classic chemotherapeutics.

Metastases ultimately are responsible for much of the suffering and mortality from cancer. A need exists to identify and target molecular and functional markers that identify metastatic cancer cells and to generate reagents for their specific inhibition.

Publications in this field include, inter alia, Li et al. Oncogene. (2009) 28(39):3442-55; United States Patent Application, 20050186571 by Ullrich et al.; United States Patent Application 20080293733 by Bearss et al.; Sun et al. Oncology. 2004; 66(6):450-7; Gustafsson et al. Clin Cancer Res. (2009) 15(14):4742-9; Wimmel et al. Eur J Cancer. 2001 37(17):2264-74; Koorstra et al. Cancer Biol Ther. 2009 8(7):618-26; Tai et al. Oncogene. (2008) 27(29):4044-55

The receptor tyrosine kinase AXL (also known as Ufo and Tyro7) belongs to a family of tyrosine receptors that includes Tyro3 (Sky) and Mer (Tyrol 2). A common ligand for AXL family is GAS6 (Growth arrest-specific protein 6). Human AXL is a 2,682-bp open reading frame capable of directing the synthesis of an 894-amino acid polypeptide. Two variant mRNAs have been characterized, transcript variant 1 may be accessed at Genbank, NM_021913.3 and transcript variant 2 may be accessed at NM_001699.4. The polypeptide sequence of the native protein is provided as SEQ ID NO:1, and specific reference may be made to the sequence with respect to amino acid modifications. Important cellular functions of GAS6/AXL include cell adhesion, migration, phagocytosis, and inhibition of apoptosis. GAS6 and AXL family receptors are highly regulated in a tissue and disease specific manner.

AXL, MER and Tyro3 are each characterized by a unique molecular structure, in that the intracellular region has the typical structure of a receptor tyrosine kinase and the extracellular domain contains fibronectin III and Ig motifs similar to cadherin-type adhesion molecules. During development, AXL, MER and Tyro3 are expressed in various organs, including the brain, suggesting that this RTK is involved in mesenchymal and neural development. In the adult, AXL, MER and Tyro3 expression is low but returns to high expression levels in a variety of tumors. GAS6 is, so far, the single, activating ligand for AXL, MER and Tyro3.

Receptor tyrosine kinases (RTK) are generally activated by ligands that promote receptor dimerization and, in turn, autophosphorylation of tyrosine residues within the cytosolic domain. Binding of signaling proteins to these phosphorylated tyrosine residues then leads to downstream signaling. AXL, MER and Tyro3 family of RTKs are unique in that they are activated by GAS6, members of the vitamin K-dependent protein family that resembles blood coagulation factors rather than typical growth factors.

### SUMMARY OF THE INVENTION

The present invention is based in part on the discovery that AXL, MER and Tyro3 and/or GAS6 related pathways are related to tumor invasion and/or metastasis. Accordingly, the present invention provides compositions and methods useful for treating tumor invasion and/or metastasis, e.g., via inhibition of AXL, MER and/or Tyro3 and/or GAS6 related pathways. In addition, the present invention provides reagents and methods useful for determining the susceptibility or likelihood of a tumor to become invasive and/or metastatic, e.g., via detecting the level of activity of AXL, MER, Tyro3 and/or GAS6.

In some embodiments, the agent useful for treating tumor invasion and/or metastasis, e.g., via inhibition of AXL, MER and Tyro3 and/or GAS6 related pathways is an inhibitor agent. In some embodiments, the inhibitor agent is selected from the group consisting of (a) an inhibitor of AXL, MER and/or Tyro3 activity, (b) an inhibitor of GAS6 activity and (c) and inhibitor of AXL, MER and/or Tyro3-GAS6 interaction, wherein the inhibitor agent is capable of binding to GAS6 with increased affinity compared to wild-type AXL, MER or Tyro3.

In some embodiments, the inhibitor agent binds to two or more epitopes on a single GAS6.

In some embodiments, at least one of the epitopes is the major or minor AXL, MER or Tyro3 binding site on GAS6.

In some embodiments, the inhibitor agent is capable of binding to the major and minor AXL, MER or Tyro3 binding sites on a single GAS6.

In some embodiments, the inhibitor agent is capable of binding to the major AXL, MER or Tyro3 binding site of GAS6 and one or more additional GAS6 epitopes on a single GAS6.

In some embodiments, the inhibitor agent is capable of binding to the minor AXL, MER or Tyro3 binding site on GAS6 and one or more additional epitopes on a single GAS6.

In some embodiments, the inhibitor agent is capable of binding two or more epitopes on a single GAS6.

In some embodiments, the inhibitor agent is capable of antagonizing the major and/or minor GAS6/receptor binding interaction, where the receptor is selected from AXL, MER and Tyro3.

In some embodiments, the inhibitor agent is capable of antagonizing the major GAS6/receptor binding interaction, where the receptor is selected from AXL, MER and Tyro3.

In some embodiments, the inhibitor agent is capable of antagonizing the minor GAS6/receptor binding interaction, where the receptor is selected from AXL, MER and Tyro3.

In some embodiments, the inhibitor agent is a polypeptide, a polypeptide-carrier fusion, a polypeptide-Fc fusion, a polypeptide-conjugate, a polypeptide-drug conjugate, an antibody, a bispecific antibody, an antibody drug conjugate, an antibody fragment, an antibody-related structure, or a combination thereof.

In some embodiments, the inhibitor agent is a natural or synthetic polypeptide.

In some embodiments, the inhibitor agent is a non-antibody polypeptide.

In some embodiments, the inhibitor agent of the present invention can include, for example but is not limited to a darpin, an avimer, an adnectin, an anticalin, an affibody, a maxibody, or other protein structural scaffold, or a combination thereof.

In some embodiments, the inhibitor agent is a polypeptide-conjugate or antibody-conjugate.

In some embodiments, the inhibitor agent is a polypeptide-polymer conjugate, where the polymer is selected from PEG, PEG-containing polymers, degradable polymers, biocompatible polymers, hydrogels, and other polymer structures or a combination thereof.

In some embodiments, the inhibitor agent is a polypeptide, wherein said polypeptide comprises a soluble AXL variant polypeptide wherein said AXL variant polypeptide lacks the AXL transmembrane domain and has at least one mutation relative to wild-type that increases affinity of the AXL polypeptide binding to GAS6.

In some embodiments, the inhibitor agent is a polypeptide, wherein said polypeptide comprises a soluble MER variant polypeptide wherein said MER variant polypeptide lacks the MER transmembrane domain and has at least one mutation relative to wild-type that increases affinity of the MER polypeptide binding to GAS6.

In some embodiments, the inhibitor agent is a polypeptide, wherein said polypeptide comprises a soluble Tyro3 variant polypeptide wherein said Tyro3 variant polypeptide lacks the Tyro3 transmembrane domain and has at least one mutation relative to wild-type that increases affinity of the Tyro3 polypeptide binding to GAS6.

In some embodiments, the inhibitor is an AXL, MER or Tyro3 variant polypeptide that inhibits binding between a wild-type AXL, MER and/or Tyro3 polypeptide and a GAS6 protein in vivo or in vitro.

In some embodiments, the polypeptide lacks a functional fibronectin (FN) domain and/or exhibits increased affinity of the AXL, MER or Tyro3 variant polypeptide binding to GAS6 compared to wild-type AXL, MER or Tyro3.

In some embodiments, the AXL, MER or Tyro3 variant polypeptide lacks the transmembrane domain, has more than one Ig1 domain and exhibits increased affinity of the AXL, MER or Tyro3 variant polypeptide binding to GAS6 as compared to wild-type AXL, MER or Tyro3.

In some embodiments, the AXL, MER or Tyro3 variant polypeptide has two Ig1 domains. In some embodiments, the polypeptide has three Ig1 domains.

In some embodiments, the AXL, MER or Tyro3 polypeptide lacks the transmembrane domain, has more than one Ig2 domain and exhibits increased affinity of the AXL, MER or Tyro3 polypeptide binding to GAS6 as compared to wild-type AXL, MER or Tyro3.

In some embodiments, the AXL, MER or Tyro3 variant polypeptide has two Ig2 domains.

In some embodiments, the polypeptide is a soluble AXL, MER or Tyro3 variant polypeptide, wherein said soluble AXL variant polypeptide lacks the AXL, MER or Tyro3 transmembrane domain, has more than one Ig1 domain, more than one Ig2 domain and exhibits increased affinity of the AXL, MER or Tyro3 variant polypeptide binding to GAS6 as compared to wild-type AXL, MER or Tyro3.

In some embodiments, the polypeptide is a soluble AXL, MER or Tyro3 variant polypeptide, wherein said soluble AXL, MER or Tyro3 variant polypeptide lacks the AXL, MER or Tyro3 transmembrane domain, lacks a functional fibronectin (FN) domain, has more than one Ig1 domain, more than one Ig2 domain, and wherein said AXL, MER or Tyro3 variant polypeptide exhibits increased affinity of the AXL, MER or Tyro3 variant polypeptide binding to GAS6 compared to wild-type AXL, MER or Tyro3.

In some embodiments, the soluble AXL, MER or Tyro3 variant polypeptide has two Ig1 domains and two Ig2 domains.

In some embodiments, the soluble AXL, MER or Tyro3 variant polypeptide has the immunoglobulin domains connected directly.

In some embodiments, the soluble AXL, MER or Tyro3 variant polypeptide has the immunoglobulin domains connected indirectly.

In some embodiments, the soluble AXL, MER or Tyro3 variant polypeptide lacks the AXL, MER or Tyro3 transmembrane domain, is capable of binding both the major and minor binding site of a single GAS6 and wherein said AXL, MER or Tyro3 variant polypeptide exhibits increased affinity of the AXL, MER or Tyro3 polypeptide binding to GAS6 as compared to wild-type AXL, MER or Tyro3.

In some embodiments, the polypeptide has one Ig1 domain and lacks a functional Ig2 domain.

In some embodiments, the polypeptide is a soluble AXL, MER or Tyro3 variant polypeptide, wherein said soluble AXL, MER or Tyro3 variant polypeptide lacks the AXL, MER or Tyro3 transmembrane domain, has one Ig1 domain, lacks a functional Ig2 domain and wherein said AXL, MER or Tyro3 variant polypeptide exhibits increased affinity of the AXL, MER or Tyro3 variant polypeptide binding to GAS6 compared to wild-type AXL, MER or Tyro3.

In some embodiments, the polypeptide is a soluble AXL, MER or Tyro3 variant polypeptide, wherein said soluble AXL, MER or Tyro3 variant polypeptide lacks the AXL, MER or Tyro3 transmembrane domain, lacks a functional fibronectin (FN) domain, has one Ig1 domain, lacks a functional Ig2 domain and wherein said AXL, MER or Tyro3 variant polypeptide exhibits increased affinity of the AXL, MER or Tyro3 variant polypeptide binding to GAS6 compared to wild-type AXL, MER or Tyro3.

In some embodiments, the AXL, MER or Tyro3 variant polypeptide is a fusion protein comprising an Fc domain.

In some embodiments, the AXL, MER or Tyro3 variant polypeptide further comprises a linker. In some embodiments, the linker comprises one or more (GLY)₄SER units.

In some embodiments, the AXL, MER or Tyro3 variant polypeptide lacks the AXL, MER or Tyro3 intracellular domain.

In some embodiments, the AXL, MER or Tyro3 variant polypeptide lacks a functional fibronectin (FN) domain and wherein said AXL, MER or Tyro3 variant polypeptide exhibits increased affinity of the polypeptide binding to GAS6 as compared to wild-type AXL, MER or Tyro3.

In some embodiments, the AXL, MER or Tyro3 variant polypeptide comprises at least one amino acid modification relative to the wild-type AXL, MER or Tyro3 sequence.

In some embodiments, the soluble AXL variant polypeptide comprises at least one amino acid modification within a region selected from the group consisting of 1) between 15-50, 2) between 60-120, and 3) between 125-135 of the wild-type AXL sequence (SEQ ID NO:1).

In some embodiments, the soluble AXL variant polypeptide comprises at least one amino acid modification at position 19, 23, 26, 27, 32, 33, 38, 44, 61, 65, 72, 74, 78, 79, 86, 87, 88, 90, 92, 97, 98, 105, 109, 112, 113, 116, 118, or 127 of the wild-type AXL sequence (SEQ ID NO: 1) or a combination thereof.

In some embodiments, the soluble AXL variant polypeptide comprises at least one amino acid modification selected from the group consisting of 1) A19T, 2) T23M, 3) E26G, 4) E27G or E27K 5) G32S, 6) N33S, 7) T38I, 8) T44A, 9) H61Y, 10) D65N, 11) A72V, 12) S74N, 13) Q78E, 14) V79M, 15) Q86R, 16) D87G, 17) D88N, 18) I90M or I90V, 19) V92A, V92G or V92D, 20) I97R, 21) T98A or T98P, 22) T105M, 23) Q109R, 24) V112A, 25) F113L, 26) H116R, 27) T118A, 28) G127R or G127E, and 29) G129E and a combination thereof.

In some embodiments, the soluble AXL variant polypeptide comprises amino acid changes relative to the wild-type AXL sequence (SEQ ID NO: 1) at the following positions: (a) glycine 32; (b) aspartic acid 87; (c) valine 92; and (d) glycine 127.

In some embodiments, the soluble AXL variant polypeptide comprises amino acid changes relative to the wild-type AXL sequence (SEQ ID NO: 1) at the following positions: (a) aspartic acid 87 and (b) valine 92.

In some embodiments, the soluble AXL variant polypeptide comprises amino acid changes relative to the wild-type AXL sequence (SEQ ID NO: 1) at the following positions: (a) glycine 32; (b) aspartic acid 87; (c) valine 92; (d) glycine 127 and (e) alanine 72.

In some embodiments, the soluble AXL variant polypeptide comprises amino acid changes relative to the wild-type AXL sequence (SEQ ID NO: 1) at the following position: alanine 72.

In some embodiments, in the soluble AXL variant polypeptide the glycine 32 residue is replaced with a serine residue, aspartic acid 87 residue is replaced with a glycine residue, valine 92 residue is replaced with an alanine residue, or glycine 127 residue is replaced with an arginine residue or a combination thereof.

In some embodiments, in the soluble AXL variant polypeptide aspartic acid 87 residue is replaced with a glycine residue or valine 92 residue is replaced with an alanine residue or a combination thereof.

In some embodiments, in the soluble AXL variant polypeptide alanine 72 residue is replaced with a valine residue.

In some embodiments, in the soluble AXL variant polypeptide glycine 32 residue is replaced with a serine residue, aspartic acid 87 residue is replaced with a glycine residue, valine 92 residue is replaced with an alanine residue, glycine 127 residue is replaced with an arginine residue or an alanine 72 residue is replaced with a valine residue or a combination thereof.

In some embodiments, the soluble AXL variant polypeptide comprises amino acid changes relative to the wild-type AXL sequence (SEQ ID NO: 1) at the following positions: (a) glutamic acid 26; (b) valine 79; (c) valine 92; and (d) glycine 127.

In some embodiments, in the soluble AXL variant polypeptide glutamic acid 26 residue is replaced with a glycine residue, valine 79 residue is replaced with a methionine residue, valine 92 residue is replaced with an alanine residue, or glycine 127 residue is replaced with an arginine residue or a combination thereof.

In some embodiments, the soluble AXL variant polypeptide comprises at least an amino acid region selected from the group consisting of amino acid region 19-437, 130-437, 19-132, 21-121, 26-132, 26-121 and 1-437 of the wild-type AXL polypeptide (SEQ ID NO: 1), and wherein one or more amino acid modifications occur in said amino acid region.

In some embodiments, the soluble AXL variant polypeptide comprises amino acid changes relative to the wild-type AXL sequence (SEQ ID NO: 1) at the following positions: (a) glycine 32; (b) aspartic acid 87; (c) alanine 72; and valine 92.

In some embodiments, in the soluble AXL variant polypeptide glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL variant polypeptide is a fusion protein comprising an Fc domain and wherein said AXL variant comprises amino acid changes relative to wild-type AXL sequence (SEQ ID NO:1) at the following positions: (a) glycine 32; (b) aspartic acid 87; (c) alanine 72; and (d) valine 92.

In some embodiments, the soluble AXL variant polypeptide of any of the preceding claims, wherein the soluble AXL polypeptide is a fusion protein comprising an Fc domain and wherein glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL variant polypeptide is a fusion protein comprising an Fc domain and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant polypeptide is a fusion protein comprising an Fc domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL variant polypeptide is a fusion protein comprising an Fc domain, lacks a functional FN domain, and wherein said AXL variant comprises amino acid changes relative to wild-type AXL sequence (SEQ ID NO:1) at the following positions: (a) glycine 32; (b) aspartic acid 87; (c) alanine 72; and (d) valine 92.

In some embodiments, the soluble AXL variant polypeptide is a fusion protein comprising an Fc domain, lacks a functional FN domain, and wherein glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL variant polypeptide is a fusion protein comprising an Fc domain, lacks a functional FN domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant polypeptide is a fusion protein comprising an Fc domain, lacks a functional FN domain, and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL variant polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72 and (d) valine 92.

In some embodiments, the soluble AXL variant polypeptide is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, and valine 92 is replaced with an alanine residue or a combination thereof.

In some embodiments, the soluble AXL variant polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant polypeptide is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL variant polypeptide of any of the preceding claims, wherein said soluble AXL variant polypeptide has an affinity of at least about 1×10⁻⁸ M, 1×10⁻⁹ M, 1×10⁻¹⁰ M, 1×10⁻¹¹ M or 1×10⁻¹² M for GAS6.

In some embodiments, the soluble AXL variant polypeptide exhibits an affinity to GAS6 that is at least about 5-fold stronger, at least about 10-fold stronger or at least about 20-fold stronger than the affinity of the wild-type AXL polypeptide.

In some embodiments, the soluble AXL variant polypeptide comprises one or more (GLY)₄SER units. In some embodiments, the linker comprises 1, 2, 3 or 5 (GLY)₄SER units.

In some embodiments, the soluble AXL variant polypeptide inhibits binding between wild-type AXL, MER and/or Tyro3 polypeptide and a GAS6 protein in vivo or in vitro.

In some embodiments, the soluble AXL variant polypeptide is a fusion polypeptide comprising an Fc domain.

In some embodiments, the present invention provides a pharmaceutical composition comprising a therapeutically effective amount of one or more soluble AXL, MER or Tyro3 variant polypeptides.

In some embodiments, the pharmaceutical composition further comprises at least one cytotoxic agent or a pharmaceutically acceptable excipient or a combination thereof.

In some embodiments, the present invention also provides methods of treating, reducing, or preventing the metastasis or invasion of a tumor in a mammalian patient, the method comprising: administering to said patient an effective dose of the inhibitor agent of the present invention. In some embodiments, the inhibitor agent is an AXL, MER or Tyro3 variant polypeptide of any of the preceding claims.

In some embodiments, the tumor for treatment is a tumor selected from the group consisting of an ovarian tumor, a breast tumor, a lung tumor, a liver tumor, a colon tumor, a gallbladder tumor, a pancreatic tumor, a prostate tumor, and glioblastoma.

### BRIEF DESCRIPTION OF THE DRAWINGS

FIG. 1. Describes the four domains of AXL and some embodiments of the various combinations of AXL-Fc constructs that have been made and tested.

FIG. 2. Describes some embodiments of the various combinations of monovalent AXL-Fc constructs.

FIG. 3. Engineering Axl variants with improved binding to Gas6. a, Axl extracellular domain consists of two immunoglobulin-like (Ig) domains containing high and low affinity Gas6 binding sites, followed by two fibronectin type III domains. Binding of Gas6 to Axl leads to receptor dimerization and activation of downstream signaling. Soluble Axl Ig1 (sAxl) sequesters Gas6, preventing activation of the Axl signaling cascade. b, Overlaid flow cytometry dot plots representing binding of yeast-displayed wild-type Axl Ig1 (red) and unsorted Axl library (blue) to 10 nM Gas6 (y-axis), and expression levels on the yeast cell surface (x-axis). c, Flow cytometry histograms of the initial yeast-displayed Axl Ig1 library and intermediate sort products compared to wild-type Axl Ig1 (gray), measuring binding to 0.5 nM Gas6 (top row) and persistent Gas6 binding after 30 h incubation with excess competitor (bottom row). MYD1 is also included for comparison. For clarity, only the gated population of yeast expressing Axl is shown. d, Binding affinities of wild-type Axl Ig1, MYD1, and Axl^{nb} as determined by KinExA. e, Binding affinities to Gas6 of every permutation of the four mutations found in MYD1.

FIG. 4. Structural basis for the affinity enhancement of MYD1. a, Gas6/MYD1 co-complex showing overall architecture and 2:2 stoichiometry. b, MYD1 Ig1 and Gas6 LG1 domains showing the location of the four mutations in Axl with respect to the major binding site which lies at the interface of these two domains. c, Side chain interactions gained (green dashes) and lost (red dashes) in Gas6/MYD1 complex compared to the wild-type complex (PDB ID 2C5D). An ordered water molecule (red sphere contacting Q94) is observed at the Gas6/MYD1 interface that is not present in the wild-type structure. Wild-type side chains are overlaid in transparent gray for comparison. The color of outlined ovals matches the shaded patches on the ribbon model (center) to indicate the location of each highlighted region. d, Local reorganization of side chains around V92A, exemplified by R48 and Q94, forms an elongated groove on MYD1 at the binding interface that allows reorientation of T457 on Gas6. e, Reorientation of T457 results in capping of the N-terminus of Helix A. f, Capping stabilizes Helix A, as seen by B-factor analysis (see Methods), where a value <0 indicates that the residue is more stable compared to average stability of the entire structure.

FIG. 5. MYD1 inhibits tumor growth and metastasis. a, Binding affinities of Axl Fc fusions to human and mouse Gas6. b, Proposed model of multivalent binding, where both arms of the Fc fusion contact a single molecule of Gas6. c, Amount of free Gas6 in serum of mice 12 hours after administration of a single dose of MYD1 Fc. d, Time course showing free Gas6 levels in serum of mice following a 1 mg/kg dose of MYD1 Fc. e-h, Anti-tumor activity of MYD1 Fc in the 4T1 breast cancer model. e, Primary tumor volume over time, P values represent comparison of Axl^{nb} to both MYD1 treatment groups. f, Quantification of proliferation in primary tumors as measured by Ki67 staining. g, Amount of lung metastases as quantified by ex vivo bioluminescent imaging. h, Representative bioluminescent images of lungs and spleens from each treatment group. i, Tumor burden in the skov3.ip ovarian tumor model as measured by number of metastases in peritoneal cavity (left) and overall weight of tumor tissue (right). j, Representative images of mice from each treatment group with arrows indicating metastases. k-l, MYD1 Fc inhibition of metastasis in OVCAR8 ovarian tumor model (k) and PDA-1 pancreatic tumor model (I) as determined by reduced weight of tumor tissue. Error bars represent ±s.e.m., n=6-12 mice per group, *P<0.05; **P<0.01; ***P<0.001; ****P<0.0001.

FIG. 6. Enhanced anti-metastatic activity of second-generation Axl variant MYD1-72. a, Binding affinities of Gas6 to Axl A72V constructs. b, Overlay of MYD1 (orange/gray) and MYD1-72 (green) co-complex structures. No significant backbone changes were observed between the structures, including on Helix A and in the loop containing residue 72 (r.m.s.d 0.25 Å). c, Free serum Gas6 following a single 0.5 mg/kg dose of either MYD1 Fc or MYD1-72 Fc. d, Anti-metastatic activity of MYD1-72 Fc compared to MYD1 Fc in the 4T1 model as measured by bioluminescent imaging of lung metastases in mice. Error bars represent ±s.e.m., n=11, *P<0.05.

FIG. 7. Flow cytometry dot plots showing sort gates and conditions used for Axl library screen. The first three sorting rounds were performed under equilibrium binding conditions. Screening stringency was imparted by decreasing the amount of Gas6 incubated with the library in each successive round of sorting. In the final three sorting rounds, the library was screened based on kinetic off-rate sort parameters in which high-affinity clones were isolated based on their ability to retain binding to Gas6 after incubation with excess competitor. See Methods for additional details.

FIG. 8. Binding affinity measurements of Axl Ig1 variants. a, N-curve analysis of Gas6 binding titrations for wild-type Axl Ig1, and Ig1 variants MYD1 and Axl^{nb}. Curve fits are shown as dotted black lines. N-curve analysis of Axl^{nb} could not be performed as complete binding to 1 nM of Gas6 was not attained even at micromolar concentrations of Axl^{nb}. This corresponds to a K_{d}>1 μM which is out of the measurable range of this assay. b, KinExA direct inject kinetic data for wild-type Axl Ig1 and MYD1 Ig1. c, KinExA signal tests of Protein S and Gas6 binding to MYD1 Fc coated beads. MYD1 Fc does not bind Protein S as no signal was detected after 700 μl of a 10 nM Protein S solution was passed over beads (left). In comparison, a robust signal was obtained after 100 μl of a 5 nM solution of Gas6 was flowed over the same beads.

FIG. 9. Binding affinity measurements of MYD1 Ig1 point mutants. N-curve analysis was performed using binding titrations to 5 nM, 500 pM, and 50 pM of Gas6 for each single, double, and triple mutant of MYD1 to determine the binding affinity values shown in FIG. 3e. Curve fits are shown as dotted black lines.

FIG. 10. Formation of Helix A on Gas6 after binding to Axl. Ribbon model of Gas6 LG1 structures when unbound (purple, PDB ID 1H30) and when bound to MYD1 (gray). The loop following strand L is disordered and not resolved in the Gas6 structure, but forms a short α-helix upon binding to MYD1. For reference, the side chain of T457 is shown in sticks in both structures. Structuring of this loop upon engagement with MYD1 is consistent with that observed for binding to wild-type Axl.

FIG. 11. Binding affinity measurements of Axl Fc fusions. a, N-curve analysis to determine binding affinity of Gas6 to wild-type Axl, MYD1 and MYD1^{-minor} Fc fusions. b, Direct inject kinetic data to determine the kₒₙ of binding interactions. Curve fits are shown as dotted black lines.

FIG. 12. Anti-tumor activity of MYD1 Fc in the 4T1 breast cancer model. Multiple independent metrics established the efficacy of MYD1 Fc in an aggressive metastatic breast cancer model. a, Primary tumor weight at the conclusion of the study. b, Metastatic burden in lungs as measured by quantitative PCR. c, Representative images of immunohistochemistry detection of Ki67 (proliferation marker) in primary tumor tissue. Brown cytoplasmic staining indicates proliferating cells; scale bar is 50 μm. d, Weight of mice in each treatment group over the course of the study showed no significant differences. Error bars represent ±s.e.m., n=11-12 mice per group, **P<0.01; ***P<0.001.

FIG. 13. Toxicology study after repeat dosing of MYD1 Fc. Mice were administered a 10 mg/kg dose of MYD1 Fc twice a week for four weeks (8 total doses). Weight of mice in each treatment group over the course of the study showed no significant differences. See Table 5 for additional toxicology results.

FIG. 14. Binding affinity measurements of MYD1-72 variants. a, N-curve analysis to determine binding affinity of Gas6 to A72V Ig1, MYD1-72 Ig1 and MYD1-72 Fc. Titrations using concentrations lower than 15 pM Gas6 were not used as robust signal-to-noise could not be obtained below this level. b, Direct inject kinetic data to determine the kₒₙ of binding interactions. Curve fits are shown as dotted black lines.

FIG. 15. Enhanced anti-tumor activity of MYD1-72 Fc in the 4T1 breast cancer model. a, Primary tumor weight at conclusion of the study was significantly lower for MYD1-72 treatment group compared to MYD1 Fc. b, Weight of mice in each treatment group over the course of the study showed no statistically significant differences. Error bars represent s.e.m., n=11-12 mice per group). *P<0.05.

### DEFINITIONS

In the description that follows, a number of terms conventionally used in the field of cell culture are utilized extensively. In order to provide a clear and consistent understanding of the specification and claims, and the scope to be given to such terms, the following definitions are provided.

“Inhibitors,” “activators,” and “modulators” of AXL on metastatic cells or its ligand GAS6 are used to refer to inhibitory, activating, or modulating molecules, respectively, identified using in vitro and in vivo assays for receptor or ligand binding or signaling, e.g., ligands, receptors, agonists, antagonists, and their homologs and mimetics.

The terms “polypeptide,” “peptide” and “protein” are used interchangeably herein to refer to a polymer of two or more amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms “antibody” and “antibodies” are used interchangeably herein and refer to a polypeptide capable of interacting with and/or binding to another molecule, often referred to as an antigen. Antibodies can include, for example “antigen-binding polypeptides” or “target-molecule binding polypeptides.” Antigens of the present invention can include for example any polypeptides described in the present invention.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. All single letters used in the present invention to represent amino acids are used according to recognized amino acid symbols routinely used in the field, e.g., A means Alanine, C means Cysteine, etc. An amino acid is represented by a single letter before and after the relevant position to reflect the change from original amino acid (before the position) to changed amino acid (after position). For example, A19T means that amino acid alanine at position 19 is changed to threonine.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer, including without limitation, adenocarcinoma of the ovary or prostate, breast cancer, glioblastoma, etc., including those who have undergone or are candidates for resection (surgery) to remove cancerous tissue. Subjects may be human, but also include other mammals, particularly those mammals useful as laboratory models for human disease, e.g. mouse, rat, etc.

The term “tumor,” as used herein, refers to all neoplastic cell growth and proliferation, whether malignant or benign, and all pre-cancerous and cancerous cells and tissues.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. In general, cells of interest for detection, analysis, classification, or treatment in the present application include precancerous (e.g., benign), malignant, pre-metastatic, metastatic, and non-metastatic cells. Examples of cancer include but are not limited to, ovarian cancer, glioblastoma, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body. Therefore, the present invention contemplates a method of determining the risk of further growth of one or more cancerous tumors in an organ or body part which is not directly connected to the organ of the original cancerous tumor and/or any steps in a process leading up to that growth.

Depending on the nature of the cancer, an appropriate patient sample is obtained. As used herein, the phrase “cancerous tissue sample” refers to any cells obtained from a cancerous tumor. In the case of solid tumors which have not metastasized, a tissue sample from the surgically removed tumor will typically be obtained and prepared for testing by conventional techniques.

The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

The term “diagnosis” is used herein to refer to the identification of a molecular or pathological state, disease or condition, such as the identification of a molecular subtype of breast cancer, prostate cancer, or other type of cancer.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

As used herein, the terms “treatment,” “treating,” and the like, refer to administering an agent, or carrying out a procedure (e.g., radiation, a surgical procedure, etc.), for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, covers any treatment of any metastatic tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. In tumor (e.g., cancer) treatment, a therapeutic agent may directly decrease the metastasis of tumor cells.

Treating may refer to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with neoplasia, e.g., tumor or cancer. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

According to the present invention, the first therapeutic can be any suitable therapeutic agent, e.g., cytotoxic agents. One exemplary class of cytotoxic agents are chemotherapeutic agents, e.g., they can be combined with treatment to inhibit AXL or GAS6 signaling. Exemplary chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, saproin, tamoxifen, taxol, topotecan hydrochloride, trastuzumab, vinblastine, vincristine and vinorelbine tartrate. For ovarian cancer treatment, a preferred chemotherapeutic agent with which an AXL or GAS6 signaling inhibitor can be combined is paclitaxel (Taxol™).

Other combination therapies are radiation, surgery, and hormone deprivation (Kwon et al., Proc. Natl. Acad. Sci U.S.A., 96: 15074-9, 1999). Angiogenesis inhibitors can also be combined with the methods of the invention.

“Concomitant administration” of a known cancer therapeutic drug with a pharmaceutical composition of the present invention means administration of the drug and AXL inhibitor at such time that both the known drug and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention.

As used herein, the phrase “disease-free survival,” refers to the lack of such tumor recurrence and/or spread and the fate of a patient after diagnosis, with respect to the effects of the cancer on the life-span of the patient. The phrase “overall survival” refers to the fate of the patient after diagnosis, despite the possibility that the cause of death in a patient is not directly due to the effects of the cancer. The phrases, “likelihood of disease-free survival”, “risk of recurrence” and variants thereof, refer to the probability of tumor recurrence or spread in a patient subsequent to diagnosis of cancer, wherein the probability is determined according to the process of the invention.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

### DETAILED DESCRIPTION

AXL, MER and Tyro3 are the three receptor protein tyrosine kinases whose ligand is GAS6. As such, the present invention is based in part on the discovery of inhibitor agents that inhibit and/or antagonize the interaction of the wild-type AXL, MER and/or Tyro3 receptor with the GAS6 ligand.

According to the present invention, such an inhibitor agent can be selected from (a) an inhibitor of AXL, MER and/or Tyro3 activity, (b) an inhibitor of GAS6 activity and (c) an inhibitor of AXL, MER and/or Tyro3-GAS6 interaction, wherein the inhibitor agent is capable of binding to GAS6 with increased affinity compared to wild-type AXL, MER and/or Tyro3.

In some embodiments, the inhibitor agent binds to two or more epitopes on a single GAS6 molecule. The two or more epitopes can include at least one of the major and/or minor AXL, MER and/or Tyro3 binding site on GAS6. In some embodiments, the epitopes are separate or distinct epitopes. In some embodiments the epitopes overlap. In some embodiments, the epitopes do not overlap. In some embodiments, the epitopes are adjacent. In some embodiments, the epitopes are not adjacent. In some embodiments, the epitopes include the major and/or minor AXL, MER and/or Tyro3 binding site on GAS6. These GAS6 epitopes of the present invention, and to which the inhibitor agents of the present invention bind, can be located on one or more GAS6 molecules. In some embodiments, the epitopes are located on a single GAS6 molecule.

In some embodiments, the inhibitor agent is capable of binding to the major and/or minor AXL, MER and/or Tyro3 binding sites on a single GAS6. In some embodiments, the inhibitor agent is capable of binding the major AXL, MER and/or Tyro3 binding site of GAS6 and one or more additional GAS6 epitopes. In other embodiments, the inhibitor agent is capable of binding to the AXL, MER and/or Tyro3 minor binding site on GAS6 and one or more additional epitopes. In some other embodiments, the inhibitor agent is capable of binding two or more distinct epitopes on GAS6. The additional GAS6 epitopes can include any epitopes on GAS6 which lead to increased affinity and/or increased avidity of the inhibitor agent binding to GAS6 as compared to wild-type AXL, MER and/or Tyro3. In some embodiments, the AXL, MER and/or Tyro3 variant polypeptides of the present invention bind two epitopes on a single GAS6 molecule. In some embodiments, the two epitopes are the major and minor AXL, MER and/or Tyro3 binding sites.

According to the invention, GAS6 receptors include AXL, MER and Tyro3. The inhibitor agents of the present invention can also in some embodiments antagonize the major and/or minor GAS6/receptor interaction. In some embodiments, the inhibitor agent is capable of antagonizing the major and/or minor GAS6/receptor binding interaction. In other embodiments, the inhibitor agent is capable of antagonizing the major GAS6/receptor binding interaction (e.g., interfering with and/or inhibiting the major GAS6/receptor binding interaction). In some embodiments, the inhibitor agent is capable of antagonizing the minor GAS6/receptor binding interaction (e.g., interfering with and/or inhibiting the minor GAS6/receptor binding interaction).

Inhibitor agents can also include for example protein scaffolds (i.e., smaller proteins that are capable of achieving comparable affinity and specificity using molecular structures that can be for example one-tenth the size of full antibodies).

The inhibitor agents can also include non-antibody polypeptides. In some embodiments, the inhibitor agent is a non-antibody polypeptide. In some embodiments, the non-antibody polypeptide can include but is not limited to peptibodies, darpins, avimers, adnectins, anticalins, affibodies, maxibodies, or other protein structural scaffold, or a combination thereof.

In some embodiments the inhibitor agent provided by the present invention is an AXL, MER and/or Tyro3 variant polypeptide, e.g., an AXL, MER and/or Tyro3 variant polypeptide that has a binding activity to GAS6 that is substantially equal to or better than the binding activity of a wild-type AXL, MER and/or Tyro3 polypeptide. In some embodiments of the present invention, the AXL, MER and/or Tyro3 variant polypeptides are utilized as therapeutic agents.

The AXL protein, with reference to the native sequence of SEQ ID NO: 1, comprises an immunoglobulin (Ig)-like domain from residues 27-128, a second Ig-like domain from residues 139-222, fibronectin type 3 domains from residues 225-332 and 333-427, intracellular domain from residues 473-894 including tyrosine kinase domain. The tyrosine residues at 779, 821 and 866 become autophosphorylated upon receptor dimerization and serve as docking sites for intracellular signaling molecules. The native cleavage site to release the soluble form of the polypeptide lies at residues 437-451.

For the purposes of the invention, a soluble form of AXL (sAXL) is the portion of the polypeptide that is sufficient to bind GAS6 at a recognizable affinity, e.g., high affinity, which normally lies between the signal sequence and the transmembrane domain, i.e. generally from about SEQ ID NO: 1 residue 19-437, but which may comprise or consist essentially of a truncated version of from about residue 19, 25, 30, 35, 40, 45, 50 to about residue 132, 450, 440, 430, 420, 410, 400, 375, 350, to 321, e.g., residue 19-132. According to the methods of the present invention, SEQ ID NO:1 can be used interchangeably with amino acids 8-894 of SEQ ID NO: 1, both of which refer to the wild-type AXL sequence. In some embodiments, a soluble form of AXL lacks the transmembrane domain, and optionally the intracellular domain.

In some embodiments, the inhibitor agent is a soluble AXL variant polypeptide that lacks the AXL transmembrane domain and has at least one mutation relative to wild-type that increases affinity of the AXL polypeptide binding to GAS6 as compared to wild-type GAS6.

The MER protein, with reference to the native SEQ ID NO:2, comprises an immunoglobulin (Ig)-like domain from residues 81-186, a second Ig-like domain from residues 197-273, fibronectin type 3 domains from residues 284-379 and 383-482, intracellular domain from residues 527-999 including tyrosine kinase domain. The tyrosine residues at 749, 753, 754 and 872 become autophosphorylated upon receptor dimerization and serve as docking sites for intracellular signaling molecules.

For the purposes of the invention, a soluble form of MER (sMER) is the portion of the polypeptide that is sufficient to bind GAS6 at a recognizable affinity, e.g., high affinity, which normally lies between the signal sequence and the transmembrane domain, i.e. generally from about SEQ ID NO: 2 residue 21-526, but which may comprise or consist essentially of a truncated version In some embodiments, a soluble form of MER lacks the transmembrane domain (i.e., generally from about SEQ ID NO: 2 residue 506-526), and optionally the intracellular domain (i.e., generally from about SEQ ID NO: 2 residue 527-999).

In some embodiments, the inhibitor agent is a soluble MER variant polypeptide wherein said MER polypeptide lacks the MER transmembrane domain and has at least one mutation relative to wild-type that increases affinity of the MER polypeptide binding to GAS6 as compared to wild-type MER.

The Tyro3 protein, with reference to the native SEQ ID NO:3, comprises an immunoglobulin (Ig)-like domain from residues 41-128, a second Ig-like domain from residues 139-220, fibronectin type 3 domains from residues 225-317 and 322-413, intracellular domain from residues 451-890 including tyrosine kinase domain. The tyrosine residues at 681, 685, 686 and 804 become autophosphorylated upon receptor dimerization and serve as docking sites for intracellular signaling molecules.

For the purposes of the invention, a soluble form of Tyro3 (sTyro3) is the portion of the Tyro3 polypeptide that is sufficient to bind GAS6 at a recognizable affinity, e.g., high affinity, which normally lies between the signal sequence and the transmembrane domain, i.e. generally from about SEQ ID NO: 3 residue 41-450, but which may comprise or consist essentially of a truncated version In some embodiments, a soluble form of AXL lacks the transmembrane domain (i.e., generally from about SEQ ID NO: 3 residue 430-450), and optionally the intracellular domain (i.e., generally from about SEQ ID NO: 3 residue 451-890).

In some embodiments, the inhibitor agent is a soluble Tyro3 variant polypeptide wherein said Tyro3 polypeptide lacks the Tyro3 transmembrane domain and has at least one mutation relative to wild-type Tyro3 that increases affinity of the Tyro3 polypeptide binding to GAS6 as compared to wild-type Tyro3.

In some embodiments, the AXL, MET or Tyro3 variant polypeptide lacks the AXL, MET or Tyro3 transmembrane domain and is a soluble variant polypeptide, e.g., sAXL, sMER or sTyro3 variant polypeptide.

In some embodiments, the AXL, MER or Tyro3 variant polypeptide lacks the AXL, MER or Tyro3 intracellular domain.

In some embodiments, the inhibitor agent of the present invention inhibits binding between a wild-type AXL, MER and/or Tyro3 polypeptide and a GAS6 protein in vivo or in vitro. In some embodiments, the AXL, MER or Tyro3 variant polypeptide inhibits binding between a wild-type AXL, MER and/or Tyro3 polypeptide and a GAS6 protein in vivo or in vitro.

The inhibitor agents of the present invention can also exhibit an enhanced or better pharmacokinetic profile. In some embodiments, the enhanced or better pharmacokinetic profile includes for example but is not limited to a better absorption profile, better distribution profile, better metabolism profile, better excretion profile, better liberation profile, increased half-life, decrease half-life, faster rate of action, longer duration of effect as compared to AXL, MER and/or Tyro3 wild-type polypeptides which do not lack a transmembrane domain. One of skill in the art would understand preferred pharmacokinetic profile parameters for particular needs including for example treatment regimens, and how to appropriately implement such parameters in treatment regimens.

The wild-type AXL, MER and Tyro3 all contain two fibronectin domains. In some embodiments, the AXL, MER and Tyro3 polypeptides of the invention lack a functional fibronectin (FN) domain. Lacks or lacking a functional fibronectin domain can include but is not limited to deletion of one or both fibronectin domains and/or introducing mutations that inhibit, reduce or remove the functionality of one or both fibronectin domains, where such mutations can include for example but are not limited to substitution, deletion and insertion mutations. In some embodiments, the polypeptides of the invention have fibronectin 1 (FN1) deleted, fibronectin 2 (FN2) deleted, or FN1 and FN 2 both deleted. In some embodiments, the polypeptides of the invention have portions of FN1 mutated and/or deleted, FN2 mutated and/or deleted, or FN1 and FN2 mutated and/or deleted.

In some embodiments, the AXL, MER or Tyro3 variant polypeptide lacks a functional AXL, MER or Tyro3 fibronectin (FN) domain. In some embodiments, the AXL, MER or Tyro3 variant polypeptide exhibits increased affinity of the polypeptide binding to GAS6 as compared to wild-type AXL, MER and/or Tyro3. In some embodiments, the AXL, MER or Tyro3 variant polypeptide lacks a functional fibronectin (FN) domain also exhibits increased affinity of the polypeptide binding to GAS6 as compared to wild-type AXL, MER and/or Tyro3.

In some embodiments, the lack of a functional fibronectin domain results in increased affinity of the AXL, MER or Tyro3 polypeptide binding to GAS6. In some embodiments, the lack of a functional fibronectin domain results in an enhanced or better pharmacokinetic profile, including for example but not limited to a better absorption profile, better distribution profile, better metabolism profile, better excretion profile, better liberation profile, increased half-life, decreased half-life, faster rate of action, longer duration of effect as compared to other wild-type polypeptides or other polypeptides which do not lack a functional fibronectin domain. One of skill in the art would understand preferred pharmacokinetic profile parameters for particular needs including for example treatment regimens, and how to appropriately implement such parameters in treatment regimens.

In some embodiments, the AXL, MER or Tyro3 variant polypeptide lacks the transmembrane domain and has more than one Ig1 domain and exhibits increased affinity of the AXL, MER or Tyro3 polypeptide binding to GAS6 as compared to wild-type AXL, MER and/or Tyro3. In some embodiments, the AXL, MER or Tyro3 polypeptide has two Ig1 domains. In some embodiments, the AXL, MER or Tyro3 polypeptide has three Ig1 domains. In some embodiments, the AXL, MER or Tyro3 polypeptide has more than one Ig1 domain and/or more than one Ig2 domain. In some embodiments, the AXL, MER or Tyro3 polypeptide has two Ig2 domains. In some embodiments, the AXL, MER or Tyro3 polypeptide has two Ig1 domains and 2 Ig2 domains. In some embodiments, the AXL, MER or Tyro3 polypeptide includes for example but is not limited to one of the following Ig domain configurations, as well as any combinations or variations thereof:
Ig1
Ig1-Ig2
Ig1-Ig1
Ig1-Ig1-Ig1
Ig1-Ig2-Ig1
Ig1-Ig2-Ig1-Ig2

In some embodiments, the AXL, MER or Tyro3 polypeptide also lacks the AXL, MER or Tyro3 transmembrane domain and/or exhibits increased affinity of the AXL, MER or Tyro3 polypeptide binding to GAS6. In some embodiments, the AXL, MER or Tyro3 variant polypeptide lacks the transmembrane domain, has more than one Ig1 domain, has more than one Ig2 domain and exhibits increased affinity of the AXL, MER or Tyro3 polypeptide binding to GAS6 as compared to wild-type AXL, MER and/or Tyro3.

In some embodiments, the AXL, MER or Tyro3 has the immunoglobulin domains connected directly to one another. In some embodiments, the AXL, MER or Tyro3 has the immunoglobulin domains connected indirectly, e.g., through a linker molecule including for example any amino acid linker known in the art.

In some embodiments, the one or more AXL, MER or Tyro3 Ig1 and/or 1 or more AXL, MER or Tyro3 Ig2 domains result in an enhanced or better pharmacokinetic profile, including for example but not limited to a better absorption profile, better distribution profile, better metabolism profile, better excretion profile, better liberation profile, increased half-life, decreased half-life, faster rate of action, longer duration of effect as compared to other wild-type polypeptides or other polypeptides which do not lack a functional fibronectin domain. One of skill in the art would understand preferred pharmacokinetic profile parameters for particular needs including for example treatment regimens, and how to appropriately implement such parameters in treatment regimens.

In some embodiments, the AXL, MER or Tyro3 variant polypeptide lacks the AXL, MER or Tyro3 transmembrane domain and is capable of binding two or more epitopes on a single GAS6. In some embodiments, the AXL, MER or Tyro3 variant polypeptide lacks the AXL, MER or Tyro3 transmembrane domain and is capable of binding both the major and minor AXL, MER and/or Tyro3 binding sites on a single GAS6. In some embodiments, the binding of both the major and minor AXL, MER and/or Tyro3 binding is simultaneous. In some embodiments, the binding of both the major and minor AXL, MER and/or Tyro3 binding sites is simultaneous on a single GAS6.

The present invention also provides AXL, MER or Tyro3 variant polypeptides that do not bind two epitopes on a single GAS6 molecule. The present invention also provides AXL, MER or Tyro3 variant polypeptides that do not bind two epitopes on a single GAS6 molecule simultaneously. In some embodiments, the AXL, MER and/or Tyro3 variant polypeptide is not capable of binding two epitopes on a single GAS6, this includes for example monomeric AXL, MER and/or Tyro3 variant polypeptides. In some embodiments, the monomeric AXL, MER or Tyro3 variant polypeptide comprises one Ig1 domain. In some embodiments, the monomeric AXL, MER and/or Tyro3 variant polypeptide is an Fc fusion polypeptide that does not bind to more than one site on a single Gas6 molecule simultaneously. In some embodiments, the monomeric AXL, MER and/or Tyro3 variant polypeptide that is not capable of binding two epitopes on a single GAS6 comprises two AXL, MER and/or Tyro3 variant polypeptides each of which are not capable of binding two epitopes on a single GAS6 simultaneously. In some embodiments, the monomeric AXL, MER and/or Tyro3 variant polypeptide that is not capable of simultaneously binding two epitopes on a single GAS6 has one Ig1 domain. In some embodiments, the monomeric AXL, MER and/or Tyro3 variant polypeptide that is not capable of simultaneously binding two epitopes on a single GAS6 has an altered half-life when compared to AXL, MER and/or Tyro3 variant polypeptides that are capable of binding two epitopes on a single GAS6. In some embodiments, the polypeptide has one Ig1 domain and lacks a functional Ig2 domain. In some embodiments, the Ig1 domain comprises amino acids 1-131 of AXL (SEQ ID NO:1; or in some embodiments 8-138 of SEQ ID NO:1). In some embodiments, the polypeptide is a soluble AXL, MER or Tyro3 variant polypeptide, wherein said soluble AXL, MER or Tyro3 variant polypeptide lacks the AXL, MER or Tyro3 transmembrane domain, has one Ig1 domain, lacks a functional Ig2 domain and wherein said AXL, MER or Tyro3 variant polypeptide exhibits increased affinity of the AXL, MER or Tyro3 variant polypeptide binding to GAS6 compared to wild-type AXL, MER or Tyro3. In some embodiments, the polypeptide of any of the preceding claims, wherein the polypeptide is a soluble AXL, MER or Tyro3 variant polypeptide, wherein said soluble AXL, MER or Tyro3 variant polypeptide lacks the AXL, MER or Tyro3 transmembrane domain, lacks a functional fibronectin (FN) domain, has one Ig1 domain, lacks a functional Ig2 domain and wherein said AXL, MER or Tyro3 variant polypeptide exhibits increased affinity of the AXL, MER or Tyro3 variant polypeptide binding to GAS6 compared to wild-type AXL, MER or Tyro3.

The wild-type AXL, MER and Tyro3 all contain an Ig2 domain. In some embodiments, the AXL, MER and Tyro3 polypeptides of the invention lack a functional Ig2 domain. Lacks or lacking a functional Ig2 domain can include but is not limited to deletion of the Ig2 domain and/or introduction of mutations that inhibit, reduce or remove the functionality of the Ig2 domain, where such mutations can include for example but are not limited to substitution, deletion and insertion mutations. In some embodiments, the polypeptides of the invention lack a functional Ig2 domain. In some embodiments, the polypeptides of the invention lack a functional Ig2 domain and have a wild-type AXL, MER and/or Tyro3 Ig1 domain. In some embodiments, the polypeptides of the invention lack a functional Ig2 domain and have one or more mutations in the Ig1 domain relative to the wild-type AXL, MER and/or Tyro3 Ig1 domain.

In some embodiments, the AXL, MER and/or Tyro3 variant polypeptide includes a linker. A wide variety of linkers are known in the art and any known linker can be employed with the methods of the present invention. In some embodiments, the AXL, MER or Tyro3 variant polypeptide includes one or more linkers or linker units. In some embodiments, the linker is an amino acid linker, including an amino acid sequence of 2, 3, 4 or 5 amino acids which are different that the wild-type AXL, MER and/or Tyro3 sequences. In some embodiments, the linker has 1, 2, 3, 4, 5, 6, 7, 8, 9, 10 or more units. In some embodiments, the linker is (GLY)₄SER (SEQ ID NO:10). In some embodiments, the linker has 1, 2, 3, 4, 5, 6, 7, 8, 9, 10 or more (GLY)₄SER units. In some embodiments, the linker has 1, 2, 3 or 5 (GLY)₄SER units. In some embodiments, the linkers are between the AXL, MER or Tyro3 variant polypeptide and the Fc portion of a fusion polypeptide. In some embodiments, the linkers are between the AXL, MER or Tyro3 variant polypeptide and the Fc portion of a fusion polypeptide and the AXL, MER or Tyro3 variant polypeptide lacks a functional fibronectin domain.

In some embodiments, AXL, MER and/or Tyro3 variant polypeptides of the present invention also include one or more amino acid modifications within the soluble form of wild-type AXL, MER and/or Tyro3, e.g., one or more amino acid modifications that increase its affinity for GAS6. According to the present invention, amino acid modifications include any naturally occurring or man-made amino acid modifications known or later discovered in the field. In some embodiments, amino acid modifications include any naturally occurring mutation, e.g., substitution, deletion, addition, insertion, etc. In some other embodiments, amino acid modifications include replacing existing amino acid with another amino acid, e.g., a conservative equivalent thereof. In yet some other embodiments, amino acid modifications include replacing one or more existing amino acids with non-natural amino acids or inserting one or more non-natural amino acids. In still some other embodiments, amino acid modifications include at least 1, 2, 3, 4, 5, or 6 or 10 amino acid mutations or changes.

In some exemplary embodiments, one or more amino acid modifications can be used to alter properties of the soluble form of AXL, MER and/or Tyro3 e.g., affecting the stability, binding activity and/or specificity, etc. Techniques for in vitro mutagenesis of cloned genes are known. Examples of protocols for scanning mutations may be found in Gustin et al., Biotechniques 14:22 (1993); Barany, Gene 37:111-23 (1985); Colicelli et al., Mol Gen Genet 199:537-9 (1985); and Prentki et al., Gene 29:303-13 (1984). Methods for site specific mutagenesis can be found in Sambrook et al., Molecular Cloning: A Laboratory Manual, CSH Press 1989, pp. 15.3-15.108; Weiner et al., Gene 126:35-41 (1993); Sayers et al., Biotechniques 13:592-6 (1992); Jones and Winistorfer, Biotechniques 12:528-30 (1992); Barton et al., Nucleic Acids Res 18:7349-55 (1990); Marotti and Tomich, Gene Anal Tech 6:67-70 (1989); and Zhu Anal Biochem 177:120-4 (1989).

In some embodiments, AXL variant polypeptides of the present invention include one or more amino acid modifications within one or more regions of residue 18 to 130, residue 10 to 135, residue 15 to 45, residue 60 to 65, residue 70 to 80, residue 85 to 90, residue 91 to 99, residue 104 to 110, residue 111 to 120, residue 125 to 130, residue 19 to 437, residue 130 to 437, residue 19 to 132, residue 21 to 132, residue 21 to 121, residue 26 to 132, or residue 26 to 121 of wild-type AXL (SEQ ID NO: 1). In some other embodiments, AXL variant polypeptides of the present invention include one or more amino acid modifications within one or more regions of residue 20 to 130, residue 37 to 124 or residue 141 to 212 of wild-type AXL (SEQ ID NO: 1). In yet some other embodiments, AXL polypeptide variants of the present invention include one or more amino acid modifications at one or more positions of position 19, 23, 26, 27, 32, 33, 38, 44, 61, 65, 72, 74, 78, 79, 86, 87, 88, 90, 92, 97, 98, 105, 109, 112, 113, 116, 118, 127, or 129 of wild-type AXL (SEQ ID NO: 1).

In yet some other embodiments, AXL polypeptide variants of the present invention include one or more amino acid modifications including without any limitation 1) A19T, 2) T23M, 3) E26G, 4) E27G or E27K, 5) G32S, 6) N33S, 7) T38I, 8) T44A, 9) H61Y, 10) D65N, 11) A72V, 12) S74N, 13) Q78E, 14) V79M, 15) Q86R, 16) D87G, 17) D88N, 18) I90M or I90V, 19) V92A, V92G or V92D, 20) 197R, 21) T98A or T98P, 22) T105M, 23) Q109R, 24) V112A, 25) F113L, 26) H116R, 27) T118A, 28) G127R or G127E, and 29) E129K and a combination thereof.

In yet some other embodiments, AXL variant polypeptides of the present invention include one or more amino acid modifications at position 32, 87, 92, or 127 of wild-type AXL (SEQ ID NO: 1) or a combination thereof, e.g., G32S; D87G; V92A and/or G127R. In yet some other embodiments, AXL polypeptide variants of the present invention include one or more amino acid modifications at position 26, 79, 92, 127 of wild-type AXL (SEQ ID NO: 1) or a combination thereof, e.g., E26G, V79M; V92A and/or G127E. In yet some other embodiments, AXL variant polypeptides of the present invention include one or more amino acid modifications at position 32, 87, 92, 127 and/or 72 of wild-type AXL (SEQ ID NO: 1) or a combination thereof, e.g., G32S; D87G; V92A; G127R and/or A72V. In yet some other embodiments, AXL variant polypeptides of the present invention include one or more amino acid modifications at position 87, 92 and/or 127 of wild-type AXL (SEQ ID NO: 1) or a combination thereof, e.g., D87G; V92A; and/or G127R. In yet some other embodiments, AXL variant polypeptides of the present invention include one or more amino acid modifications at position 32, 92, and/or 127 of wild-type AXL (SEQ ID NO: 1) or a combination thereof, e.g., G32S; V92A; and/or G127R. In yet some other embodiments, AXL variant polypeptides of the present invention include one or more amino acid modifications at position 32, 87 and/or 127 of wild-type AXL (SEQ ID NO: 1) or a combination thereof, e.g., G32S; D87G; and/or G127R. In yet some other embodiments, AXL polypeptide variants of the present invention include one or more amino acid modifications at position 32, 87 and/or 92 of wild-type AXL (SEQ ID NO: 1) or a combination thereof, e.g., G32S; D87G; and/or V92A. In yet some other embodiments, AXL variant polypeptides of the present invention include one or more amino acid modifications at position 26, 79, 92, 127 of wild-type AXL (SEQ ID NO: 1) or a combination thereof, e.g., E26G, V79M; V92A and/or G127E. In yet some other embodiments, AXL variant polypeptides of the present invention include one or more amino acid modifications at position 87 and 92 of wild-type AXL (SEQ ID NO: 1) or a combination thereof, e.g., D87G and V92A. In yet some other embodiments, AXL variant polypeptides of the present invention include at least one amino acid modification at position 72 of wild-type AXL (SEQ ID NO: 1), e.g., A72V.

According to the present invention, the inhibitor agent can include but is not limited to a polypeptide, a polypeptide-carrier fusion, a polypeptide-Fc fusion, polypeptide-conjugate, a polypeptide-drug conjugate, an antibody, a bispecific antibody, an antibody-drug conjugate, an antibody fragment, an antibody-related structure, or a combination thereof.

The inhibitor agents of the present invention can include peptides or polypeptides. The peptides and polypeptides of the present invention can include natural and/or synthetic polypeptides. Synthetic polypeptides and methods of making synthetic polypeptides are well known in the art and any known methods for making synthetic polypeptides can be employed with the methods of the present invention. In some embodiments, the inhibitor agent is a natural or synthetic polypeptide. In some embodiments, the inhibitor agent is a natural or synthetic polypeptide-fusion. In some embodiments, the inhibitor agent is a natural or synthetic polypeptide-Fc fusion. In some embodiments the natural or synthetic polypeptide-fusion is a fusion with another protein structural class or scaffold or a natural or synthetic polypeptide-fusion with a polymer or hydrogel or related structure.

According to the present invention, the AXL, MER or Tyro3 variant polypeptides of the present invention can be further modified, e.g., joined to a wide variety of other oligopeptides or proteins for a variety of purposes. For instance, various post-translation or post-expression modifications can be carried out with respect to AXL, MER or Tyro3 variant polypeptides of the present invention. For example, by employing the appropriate coding sequences, one may provide farnesylation or prenylation. In some embodiments, the AXL, MER or Tyro3 variant polypeptides of the present invention can be PEGylated, where the polyethyleneoxy group provides for enhanced lifetime in the blood stream. The AXL, MER or Tyro3 variant polypeptides of the present invention can also be combined with other proteins, such as the Fc of an IgG isotype, which can be complement binding, with a toxin, such as ricin, abrin, diphtheria toxin, or the like, or with specific binding agents that allow targeting to specific moieties on a target cell. The inhibitor agents of the present invention can include polypeptide conjugates and antibody-conjugates. In some embodiments, the inhibitor agent is a polypeptide-conjugate or antibody-conjugate. In some embodiments, the polypeptide conjugate is a drug conjugate. In some embodiments, the peptide or polypeptide conjugate is an antibody-drug conjugates. In some embodiments, the polypeptide conjugate is a polymer conjugate. Polymers of the present invention include but are not limited to PEG, PEG-containing polymers, degradable polymers, biocompatible polymers, hydrogels, as well as other polymer structures that could be conjugated to a polypeptide, and can include combinations thereof.

In some embodiments, the AXL, MER or Tyro3 variant polypeptide of the present invention is a fusion protein, e.g., fused in frame with a second polypeptide. In some embodiments, the second polypeptide is capable of increasing the size of the fusion protein, e.g., so that the fusion protein will not be cleared from the circulation rapidly. In some other embodiments, the second polypeptide is part or whole of Fc region. In some other embodiments, the second polypeptide is any suitable polypeptide that is substantially similar to Fc, e.g., providing increased size and/or additional binding or interaction with Ig molecules. In yet some other embodiments, the second polypeptide is part or whole of an albumin protein, e.g., a human serum albumin protein. In some embodiments, the second polypeptide is a protein or peptide that binds to albumin.

In some other embodiments, the second polypeptide is useful for handling the AXL, MER or Tyro3 variant polypeptides, e.g., purification of AXL, MER or Tyro3 variant polypeptides or for increasing stability in vitro or in vivo. For example, AXL, MER or Tyro3 variant polypeptides of the present invention can be combined with parts of the constant domain of immunoglobulins (IgG), resulting in chimeric or fusion polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. EP A 394,827; Traunecker et al., Nature, 331: 84-86, 1988. Fusion proteins having disulfide-linked dimeric structures (due to the IgG) can also be more efficient in binding and neutralizing other molecules, than the monomeric secreted protein or protein fragment alone. Fountoulakis et al., J. Biochem. 270: 3958-3964, 1995.

In yet some other embodiments, the second polypeptide is a marker sequence, such as a peptide which facilitates purification of the fused polypeptide. For example, the marker amino acid sequence can be a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86: 821-824, 1989, for instance, hexa-histidine provides for convenient purification of the fusion protein. Another peptide tag useful for purification, the “HA” tag, corresponds to an epitope derived from the influenza hemagglutinin protein. Wilson et al., Cell 37: 767, 1984.

In still some other embodiments, the second polypeptide is an entity useful for improving the characteristics of AXL, MER or Tyro3 polypeptide variants of the present invention. For instance, a region of additional amino acids, particularly charged amino acids, may be added to the N-terminus of the polypeptide to improve stability and persistence during purification from the host cell or subsequent handling and storage. Also, peptide moieties may be added to the AXL, MER or Tyro3 polypeptide variants of the present invention to facilitate purification and subsequently removed prior to final preparation of the polypeptide. The addition of peptide moieties to facilitate handling of polypeptides are familiar and routine techniques in the art.

In still yet some embodiments, AXL, MER or Tyro3 variant polypeptides of the present invention have a binding activity to GAS6 that is at least equal or better than the wild-type AXL, MER or Tyro3. In some other embodiments, AXL, MER or Tyro3 variant polypeptides of the present invention has a binding activity or affinity to GAS6 that is at least 1-fold, 2-fold, 3-fold, 4-fold, 5-fold, or 6-fold greater than that of the wild-type AXL, MER or Tyro3. In some other embodiments, AXL, MER or Tyro3 polypeptide variant of the present invention has a binding activity or affinity to GAS6 of at least about 1×10⁻⁶, 1×10⁻⁷, 1×10⁻⁸ or 1×10⁻⁹ M 1×10⁻¹⁹ M, 1×10⁻¹¹ M or 1×10⁻¹²M. In yet some other embodiments, sAXL polypeptides of the present invention is capable of inhibiting, inhibit or compete with wild-type AXL binding to GAS6 either in vivo, in vitro or both. In yet some other embodiments, sAXL polypeptides of the present invention inhibit or compete with the binding of AXL S6-1, AXL S6-2, and/or AXL S6-5 (as described in WO2011/091305). In yet some other embodiments, sAXL polypeptides of the present invention inhibit or compete with the binding of any sAXL variant as described in WO2011/091305.

The inhibitor agents of the present invention bind to GAS6 with increased affinity. In some embodiments, the AXL, MER or Tyro3 variant polypeptide exhibits increased affinity of the AXL, MER or Tyro3 polypeptide binding to GAS6 as compared to wild-type AXL, MER or Tyro3. In some embodiments, AXL, MER or Tyro3 variant polypeptide exhibits an affinity to GAS6 that is at least about 5-fold stronger, at least about 10-fold stronger or at least about 20-fold stronger, 50-fold stronger, 100-fold stronger or at least 200-fold stronger, etc. than the affinity of the wild-type AXL, MER or Tyro3 polypeptide. In some embodiments, the soluble AXL has a about a 115-fold stronger affinity to GAS6 than the affinity of the wild-type AXL polypeptide.

The ability of a molecule to bind to GAS6 can be determined, for example, by the ability of the putative ligand to bind to GAS6 coated on an assay plate. In one embodiment, the binding activity of AXL, MER or Tyro3 variant polypeptides of the present invention to a GAS6 can be assayed by either immobilizing the ligand, e.g., GAS6 or the AXL, MER or Tyro3 variant polypeptides. For example, the assay can include immobilizing GAS6 fused to a His tag onto Ni-activated NTA resin beads. Agents can be added in an appropriate buffer and the beads incubated for a period of time at a given temperature. After washes to remove unbound material, the bound protein can be released with, for example, SDS, buffers with a high pH, and the like and analyzed.

In still yet other embodiments, AXL, MER or Tyro3 variant polypeptides of the present invention has a better thermal stability than the thermal stability of a wild-type AXL. In some embodiments, the melting temperature of AXL, MER or Tyro3 variant polypeptides of the present invention is at least 5° C., 10° C., 15° C., or 20° C. higher than the melting temperature of a wild-type AXL.

According to the present invention, AXL, MER or Tyro3 variant polypeptides of the present invention can also include one or more modifications that do not alter primary sequences of the AXL, MER or Tyro3 variant polypeptides of the present invention. For example, such modifications can include chemical derivatization of polypeptides, e.g., acetylation, amidation, carboxylation, etc. Such modifications can also include modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. In some embodiments, AXL, MER or Tyro3 polypeptide variants of the present invention include AXL, MER or Tyro3 variant polypeptides having phosphorylated amino acid residues, e.g. phosphotyrosine, phosphoserine, or phosphothreonine.

In some other embodiments, AXL, MER or Tyro3 variant polypeptides of the present invention include AXL, MER or Tyro3 variant polypeptides further modified to improve their resistance to proteolytic degradation or to optimize solubility properties or to render them more suitable as a therapeutic agent. For example, AXL, MER or Tyro3 polypeptide variants of the present invention further include analogs of AXL, MER or Tyro3 variant polypeptides containing residues other than naturally occurring L-amino acids, e.g. D-amino acids or non-naturally occurring synthetic amino acids. D-amino acids may be substituted for some or all of the amino acid residues.

In yet some other embodiments, AXL, MER or Tyro3 variant polypeptides of the present invention include at least two same or different AXL, MER or Tyro3 variant polypeptides linked covalently or non-covalently. For example, in some embodiments, AXL, MER or Tyro3 polypeptide variants of the present invention include two, three, four, five, or six same or different AXL, MER or Tyro3 variant polypeptides linked covalently, e.g., so that they will have the appropriate size, but avoiding unwanted aggregation.

According to the present invention, AXL, MER or Tyro3 variant polypeptides of the present invention can be produced by any suitable means known or later discovered in the field, e.g., produced from eukaryotic or prokaryotic cells, synthesized in vitro, etc. Where the protein is produced by prokaryotic cells, it may be further processed by unfolding, e.g. heat denaturation, DTT reduction, etc. and may be further refolded, using methods known in the art.

The AXL, MER or Tyro3 variant polypeptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

The AXL, MER or Tyro3 variant polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Methods which are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized. One of skill in the art can readily utilize well-known codon usage tables and synthetic methods to provide a suitable coding sequence for any of the polypeptides of the invention. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences may be cloned and the appropriate subsequences cleaved using appropriate restriction enzymes.

The nucleic acids may be isolated and obtained in substantial purity. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome. The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

In some embodiments, the present invention provides expression vectors for in vitro or in vivo expression of one or more AXL, MER and/or Tyro3 polypeptide variants of the present invention, either constitutively or under one or more regulatory elements. In some embodiments, the present invention provides a cell population comprising one or more expression vectors for expressing AXL, MER and/or Tyro3 polypeptide variants of the present invention, either constitutively or under one or more regulatory elements.

According to another aspect of the invention, it provides isolated antibodies or fragments thereof which specifically bind to a GAS6 protein. GAS6 (growth arrest-specific 6) belongs structurally to the family of plasma vitamin K-dependent proteins. GAS6 has a high structural homology with the natural anticoagulant protein S, sharing the same modular composition and having 40% sequence identity. GAS6 has growth factor-like properties through its interaction with receptor tyrosine kinases of the TAM family; Tyro3, AXL and MER. Human GAS6 is a 678 amino acid protein that consists of a gamma-carboxyglutamate (Gla)-rich domain that mediates binding to phospholipid membranes, four epidermal growth factor-like domains, and two laminin G-like (LG) domains. The sequence of the transcript variants of human GAS6 may be accessed at Genbank at NM_001143946.1; NM_001143945.1; and NM_000820.2, respectively.

GAS6 employs a unique mechanism of action, interacting through its vitamin K-dependent GLA (gamma-carboxyglutamic acid) module with phosphatidylserine-containing membranes and through its carboxy-terminal LamG domains with the TAM membrane receptors.

According to the present invention, isolated antibodies of the present invention include any isolated antibodies with a recognizable binding specificity against GAS6. In some embodiments, isolated antibodies are partially or fully humanized antibodies. In some other embodiments, isolated antibodies are monoclonal or polyclonal antibodies. In yet some other embodiments, isolated antibodies are chimeric antibodies, e.g., with consistent regions, variable regions and/or CDR3 or a combination thereof from different sources. In yet some other embodiments, isolated antibodies are a combination of various features described herein.

According to the present invention, fragments of the isolated antibodies of the present invention include a polypeptide containing a region of the antibody (either in the context of an antibody scaffold or a non-antibody scaffold) that is sufficient or necessary for a recognizable specific binding of the polypeptide towards GAS6. In some embodiments, fragments of the isolated antibodies of the present invention include variable light chains, variable heavy chains, one or more CDRs of heavy chains or light chains or combinations thereof, e.g., Fab, Fv, etc. In some embodiments, fragments of the isolated antibodies of the present invention include a polypeptide containing a single chain antibody, e.g., ScFv. In yet some embodiments, fragments of the isolated antibodies of the present invention include variable regions only or variable regions in combination with part of Fc region, e.g., CH1 region. In still some embodiments, fragments of the isolated antibodies of the present invention include minibodies, e.g., VL-VH-CH3 or diabodies.

In some embodiments, isolated antibodies of the present invention bind to an epitope comprised in or presented by one or more amino acid regions that interact with AXL, MER and/or Tyro3. In some other embodiments, isolated antibodies of the present invention bind to an epitope comprised in or presented by one or more amino acid regions of GAS6, e.g., L295-T317, E356-P372, R389-N396, D398-A406, E413-H429, and W450-M468 of GAS6.

In yet some other embodiments, isolated antibodies of the present invention bind to an epitope comprised in or presented by one or more amino acid regions, e.g., LRMFSGTPVIRLRFKRLQPT (SEQ ID NO: 4), EIVGRVTSSGP (SEQ ID NO: 5), RNLVIKVN (SEQ ID NO: 6), DAVMKIAVA (SEQ ID NO: 7), ERGLYHLNLTVGIPFH (SEQ ID NO: 8), and WLNGEDTTIQETVVNRM (SEQ ID NO: 9).

In yet some other embodiments, isolated antibodies of the present invention bind to an epitope comprised in or presented by at least one, two, three, four, five, or six amino acids in a region of L295-T317, E356-P372, R389-N396, D398-A406, E413-H429, and W450-M468 of GAS6. In yet some other embodiments, isolated antibodies of the present invention bind to an epitope comprised in or presented by at least one, two, three, four, five or six amino acids in a region of LRMFSGTPVIRLRFKRLQPT (SEQ ID NO: 4), EIVGRVTSSGP (SEQ ID NO: 5), RNLVIKVN (SEQ ID NO: 6), DAVMKIAVA (SEQ ID NO: 7), ERGLYHLNLTVGIPFH (SEQ ID NO: 8), and WLNGEDTTIQETVVNRM (SEQ ID NO: 9).

In still some other embodiments, isolated antibodies of the present invention is capable of inhibiting, inhibits or competes with the binding between wild-type AXL, MER and/or Tyro3 or AXL, MER and/or Tyro3 polypeptide variants of the present invention and GAS6.

According to the present invention, the AXL, MER or Tyro3 variant polypeptides and isolated antibodies of the present invention can be provided in pharmaceutical compositions suitable for therapeutic use, e.g., for human treatment. In some embodiments, pharmaceutical compositions of the present invention include one or more therapeutic entities of the present invention, e.g., AXL polypeptide variants and/or isolated antibodies against GAS6 or pharmaceutically acceptable salts, esters or solvates thereof or any prodrug thereof. In some other embodiments, pharmaceutical compositions of the present invention include one or more therapeutic entities of the present invention in combination with another cytotoxic agent, e.g., another anti-tumor agent. In yet some other embodiments, pharmaceutical compositions of the present invention include one or more therapeutic entities of the present invention in combination with another pharmaceutically acceptable excipient.

In still some other embodiments, therapeutic entities of the present invention are often administered as pharmaceutical compositions comprising an active therapeutic agent, i.e., and a variety of other pharmaceutically acceptable components. (See Remington's Pharmaceutical Science, 15.sup.th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions of the present invention can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

According to yet another aspect of the invention, it provides methods for treating, reducing or preventing tumor metastasis or tumor invasion by inhibiting the AXL, MER or Tyro3 signaling pathway and/or GAS6 signaling pathway. In some embodiments, methods of the present invention include inhibiting the activity of AXL, MER, Tyro3 and/or GAS6, or the interaction between AXL, MER and/or Tyro3 and GAS6. For example, the activity of AXL, MER, Tyro3 and/or GAS6 can be inhibited at the gene expression level, mRNA processing level, translation level, post-translation level, protein activation level, etc. In some other examples, the activity of AXL, MER, Tyro3 or GAS6 can be inhibited by small molecules, biological molecules, e.g., polypeptides, polynucleotides, antibodies, antibody drug conjugates, etc. In some other examples, the activity of AXL, MER, Tyro3 or GAS6 can be inhibited by one or more AXL, MER or Tyro3 variant polypeptides or isolated antibodies of the present invention.

In yet other embodiments, methods of the present invention include administering to a subject in need of treatment a therapeutically effective amount or an effective dose of a therapeutic entity (e.g., inhibitor agent) of the present invention, e.g., an inhibitor of AXL, MER and/or Tyro3 activity or GAS6 activity or an inhibitor of interaction between AXL, MER and/or Tyro3 and GAS6. In some embodiments, effective doses of the therapeutic entity of the present invention, e.g. for the treatment of metastatic cancer, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

In some embodiments, the dosage may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

In still other embodiments, methods of the present invention include treating, reducing or preventing tumor metastasis or tumor invasion of ovarian cancer, breast cancer, lung cancer, liver cancer, colon cancer, gallbladder cancer, pancreatic cancer, prostate cancer, and/or glioblastoma.

In still yet some other embodiments, for prophylactic applications, pharmaceutical compositions or medicaments are administered to a patient susceptible to, or otherwise at risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

In still yet some other embodiments, for therapeutic applications, therapeutic entities of the present invention are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved. Typically, the response is monitored and repeated dosages are given if there is a recurrence of the cancer.

According to the present invention, compositions for the treatment of metastatic cancer can be administered by parenteral, topical, intravenous, intratumoral, oral, subcutaneous, intraarterial, intracranial, intraperitoneal, intranasal or intramuscular means. The most typical route of administration is intravenous or intratumoral although other routes can be equally effective.

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies and/or polypeptides can be administered in the form of a depot injection or implant preparation which can be formulated in such a manner as to permit a sustained release of the active ingredient. An exemplary composition comprises polypeptide at 1 mg/mL, formulated in aqueous buffer consisting of 10 mM Tris, 210 mM sucrose, 51 mM L-arginine, 0.01% polysorbate 20, adjusted to pH 7.4 with HCl or NaOH.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

Preferably, a therapeutically effective dose of the antibody compositions described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

Also within the scope of the invention are kits comprising the compositions (e.g., AXL, MER or Tyro3 variant polypeptides and formulations thereof) of the invention and instructions for use. The kit can further contain a least one additional reagent. Kits typically include a label indicating the intended use of the contents of the kit. The term label includes any writing, or recorded material supplied on or with the kit, or which otherwise accompanies the kit.

According to yet another aspect of the invention, it provides methods for determining the ability of a tumor to undergo tumor invasion and/or metastasis by detecting and/or determining the level of AXL, MER and/or Tyro3 activity or GAS6 activity in a biological sample from a subject of interest. In some embodiment, the level of AXL, MER and/or Tyro3 activity or GAS6 activity is measured by the level of mRNA expression, the level of protein expression, the level of protein activation or any suitable indicator corresponding to the activity of AXL, MER and/or Tyro3 or GAS6 either directly or indirectly. In some embodiments, the level of AXL, MER and/or Tyro3 activity or GAS6 activity in a biological sample is further compared to a predetermined level, e.g., standard level obtained by establishing normal levels or ranges of AXL, MER and/or Tyro3 activity or GAS6 activity based on a population of samples from tumors that do not develop tumor invasion or tumor metastasis or from normal tissues. For example, an increase of AXL, MER and/or Tyro3 activity or GAS6 activity over the predetermined level or standard level is indicative of a predisposition of the tumor to undergo tumor invasion or tumor metastasis.

Also within the scope of the disclosure are GAS6 inhibitor agents that stabilize Helix A of GAS6 (see examples section). A segment of Gas6 exists as a disordered loop when not bound to Axl. Upon receptor binding, structuring of the loop results in the formation of a loosely formed alpha-helix (referred to herein as “Helix A”). Helix A represents the bound conformation of the ligand. If the stability of the loosely formed helix is improved, the bound conformation is stabilized and the binding affinity is increased. We show in the examples below that high affinity AXL variants can be engineered that leverage this mechanism. In particular, AXL variants that contain the V92A mutation cause a structural change on GAS6 analogous to N-terminal helix capping resulting in the stabilization of the bound conformation of GAS6. Thus, in some embodiments, a subject GAS6 inhibitor agent stabilizes Helix A on GAS6. In some embodiments, Helix A on GAS6, involved in binding with AXL, is stabilized by a mechanism analogous to N-terminal capping upon binding of a subject GAS6 inhibitor agent to GAS6. In some embodiments, a subject GAS6 inhibitor agent is capable of inducing a conformational change of GAS6 that stabilizes Helix A of GAS6. In some embodiments, the GAS6 inhibitor agent is capable of causing a structural change of Helix A of GAS6 that stabilizes the bound conformation of the GAS6 through N-terminal capping of Helix A of GAS6. In some embodiments, the GAS6 inhibitor agent optimizes the GAS6/AXL binding interface by causing a structural change on Gas6 that stabilizes the bound conformation of the ligand through N-terminal capping of Helix A.

Also within the scope of the disclosure are methods of producing a GAS6 inhibitor agent that is capable of inducing a conformational change of GAS6 that stabilizes Helix A of GAS6, the method comprising: (a) measuring the binding affinity for GAS6 of a candidate GAS6 inhibitor agent; and (b) when the affinity for GAS6 of the candidate GAS6 inhibitor agent is greater than the affinity of wild type AXL (SEQ ID NO: 1), determining the crystal structure of GAS6 bound to the candidate GAS6 inhibitor agent to determine whether the candidate GAS6 inhibitor agent induces a conformational change of GAS6 that stabilizes Helix A of GAS6. In some embodiments, the structure (e.g., crystal structure) is determined by X-ray crystallography. Methods (e.g., X-ray crystallography) of determining the structure (e.g., crystal structure) of a complex are well known to one of ordinary skill in the art.

Also within the scope of the disclosure are methods of measuring the amount of GAS6 (e.g., the concentration of GAS6) in a sample. In some embodiments the GAS6 that is measured is free GAS6. Free GAS6 is the fraction of GAS6 that is unbound by a GAS6 binding reagent (e.g., an inhibitor, a naturally existing receptor, and the like), and thus is capable of activating AXL on a cell surface. In some embodiments, the methods quantitatively measure the amount of GAS6 in a sample.

Methods of measuring the amount of Gas6 generally employ a capture reagent with high-affinity (i.e., higher affinity than the affinity between wild type Gas6 and wild type Axl) for GAS6. In some cases, the capture reagent is immobilized, i.e., bound to a solid or semi-solid surface (e.g., a microparticle, a plastic surface, a glass surface, etc.). The term “GAS6 capture reagent,” as used herein in the context of measuring GAS6, is synonymous with the term “GAS6 inhibitor agent.” For example, a GAS6 capture reagent can be any GAS6 inhibitor agent (e.g., a variant AXL polypeptide fused to an Fc domain).

In some embodiments, the method comprises: (a) contacting a GAS6 capture reagent with a sample to produce a contacted sample, wherein the capture reagent is capable of binding to GAS6 polypeptide with increased affinity compared to wild-type AXL; and (b) measuring the amount of GAS6 polypeptide that is bound to the capture reagent. The measuring step can be performed by any convenient method for detecting binding interactions (e.g., enzyme-linked immunosorbent assay (ELISA), fluorescence resonance energy transfer (FRET), etc.).

In some cases, the step of measuring comprises contacting the contacted sample with a detectable GAS6 binding agent. The detectable GAS6 binding agent can be any binding agent (e.g., an anti-GAS6 antibody, a detectable GAS6 inhibitor agent, a fusion protein, etc.) In some cases, the detectable GAS6 binding agent is an enzyme conjugated agent (e.g., conjugated to alkaline phosphatase, conjugated to beta-galactosidase, etc.). In some cases, the detectable GAS6 binding agent is an anti-GAS6 antibody. In some such cases, the anti-GAS6 antibody can be detected by a secondary means (e.g., using a secondary antibody that is either labeled or conjugated to a detectable enzyme, etc.) In some cases, the GAS6 binding agent is detectabley labeled (e.g., conjugated to a detectable particle, e.g., a gold particle, a fluorescent particle, etc.; conjugated to a fluorescent protein, e.g., a red fluorescent protein, a green fluorescent protein, a blue fluorescent protein, etc.). In some cases, the method of measuring the amount of Gas6 is an ELISA.

In some embodiments, the method includes a washing step (i.e., a step of removing unbound GAS6 polypeptide and/or unbound GAS6 inhibitor agent). In some such cases, the method is a method of measuring the amount of Gas6 in a sample, the method comprising: (a) contacting a GAS6 inhibitor agent with the sample to produce a contacted sample, wherein the inhibitor agent is capable of producing a bound complex comprising GAS6 polypeptide bound to the GAS6 inhibitor agent, and wherein the inhibitor agent is capable of binding to GAS6 polypeptide with increased affinity compared to wild-type AXL; (b) removing unbound inhibitor agent and/or unbound Gas6 polypeptide from the contacted sample to produce a contacted washed sample; and (c) measuring the amount of bound complex present in the contacted washed sample.

Methods of measuring the amount of GAS6 (e.g., the concentration of Gas6) in a sample are useful, for example, for measuring the amount of GAS6 in a sample (e.g., serum, blood, urine, etc.) from an individual (e.g., a mammal, a human patient, etc.). Measuring free GAS6 levels in a sample can be used as a diagnostic tool because GAS6 levels correlate with many disease states (e.g., many different types of cancer). For examples of diseases and disease states associated with GAS6 (and therefore suitable for prognostic, diagnostic, and treatment methods disclosed herein), refer to patent application publications US20130189254, US20130017205, US20130108644, and WO2011091305; and application Ser. Nos. 61/737,277, and 61/737,276 (all of which are herein incorporated by reference in their entirety). The methods can therefore be used for diagnosis and prognosis (e.g., to stratisfy individuals based on GAS6 levels for the risk of acquiring a particular disease, the risk of a particular disease progressing, etc.). In some cases, in order to determine a diagnosis, prognosis or prediction, the amount of GAS6 measured from the sample is compared to a reference or control. A reference or control can be: (i) a known reference standard (i.e., a pre-measured known threshold amount (concentration) of GAS6); and/or (ii) the amount of GAS6 measured from a reference sample (e.g., a reference sample from which the amount of GAS6 is measured). The reference or control is typically a measured GAS6 amount from a sample (e.g., a body fluid or a tissue) with a known association with a particular phenotype (e.g., non-cancer patient, cancer patient, cancer patient with good prognosis, cancer patient with poor prognosis, etc.). Thus, for example, if the amount of GAS6 that is measured in an individual is higher than the reference (when the reference is a normal control reference), then the amount may be predictive of disease. On the other hand, if the reference is from a disease sample, then a measured amount of GAS6 from an individual that is similar to the reference may be predictive of disease. Likewise, if the amount of GAS6 that is measured in an individual is lower than the reference (when the reference is disease reference, i.e., from a disease sample), then the amount may be predictive that the individual is normal (e.g., does not have the disease). On the other hand, if the reference is a normal control reference, then a measured amount of GAS6 from an individual that is similar to the reference may be predictive that the individual is normal (e.g., does not have the disease).

Because GAS6 inhibitor agents (e.g., variant AXL polypeptides) are potent antagonists of the AXL receptor (e.g., the effectively bind and neutralize AXL's ligand, GAS6), they have broad therapeutic applicability (e.g., as anti-cancer drugs). The methods of measuring the amount of GAS6 (disclosed herein) can serve as a companion diagnostic to monitor the activity of GAS6 inhibitor agents administered to an individual. For example, measuring the amount of free GAS6 in a sample from an individual to whom a GAS6 inhibitor agent has been administered can provide information about the efficacy of agent administration (e.g., how well the inhibitor agent is performing in the individual), and can in some cases provide a guide for further therapy (e.g., a guide for determining a course of treatment, e.g., whether to increase, decrease, or discontinue GAS6 inhibitor agent therapy; whether to use a different GAS6 inhibitor agent; etc.). In some cases, the amount of GAS6 is measured in two or more samples obtained from the patient, where the samples are obtained at different times, thus providing the ability to monitor progress over time. In some embodiments, GAS6 is measured from a first sample obtained from an individual prior to administering a dose of GAS6 inhibitor agent, and from a second sample obtained from an individual after a dose of GAS6 inhibitor agent is administered. When the amount of GAS6 (e.g., free GAS6) decreases over time (i.e., the amount measured from the second sample is lower than the amount measured from the first sample), then the GAS6 inhibitory agent is effective (i.e., the inhibitor agent is binding free GAS6). However, if the amount of GAS6 does not decrease over time (i.e., the amount measured from the second sample is greater than or equal to the amount measured from the first sample), then the GAS6 inhibitor is not effective.

Thus, in some embodiments, the method is a method of treating an individual, the method comprising: (a) administering to the individual a GAS6 inhibitor agent that binds to GAS6 with higher affinity than wild type AXL (SEQ ID NO: 1); and (b) measuring the amount of GAS6 in a biological sample obtained from the individual, wherein the measuring is performed by a method comprising: contacting a GAS6 capture reagent with a sample to produce a contacted sample, wherein the capture reagent is capable of binding to GAS6 polypeptide with increased affinity compared to wild-type AXL; and measuring the amount of GAS6 polypeptide that is bound to the capture reagent.

In some embodiments, the method is a method of determining the efficacy of a GAS6 inhibitor agent, the method comprising: measuring the amount of GAS6 in a biological sample obtained from an individual undergoing treatment that comprises the administration of a GAS6 inhibitor agent, wherein the measuring is performed by a method comprising: contacting a GAS6 capture reagent with a sample to produce a contacted sample, wherein the capture reagent is capable of binding to GAS6 polypeptide with increased affinity compared to wild-type AXL; and measuring the amount of GAS6 polypeptide that is bound to the capture reagent. In some cases, the method further comprises, prior to the step of measuring, administering to the individual a GAS6 inhibitor agent that binds to GAS6 with higher affinity than wild type AXL (SEQ ID NO: 1).

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, examples will be described to illustrate parts of the invention. It is also understood that the terminology used herein is for the purposes of describing particular embodiments.

### EXPERIMENTAL

### Example 1

### Affinities of Various Axl Fc Constructs

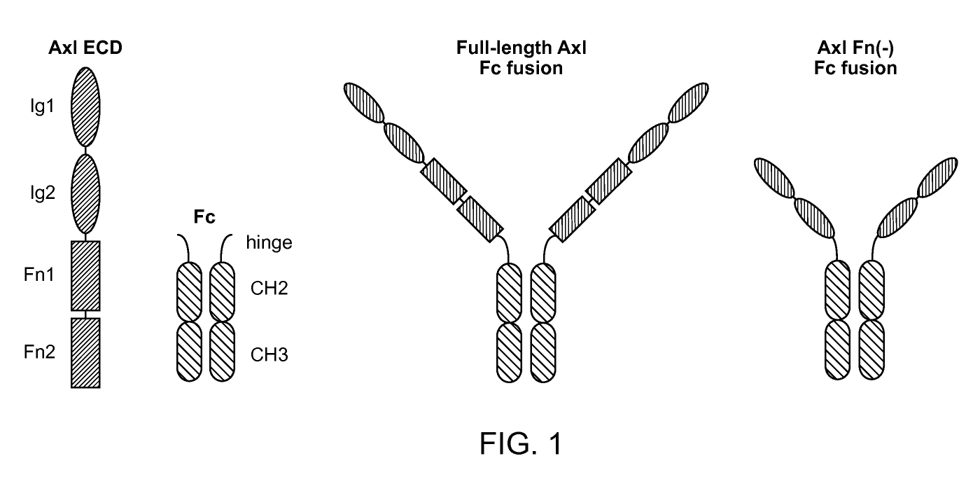

FIG. 1 shows the four domains of AXL and the various combinations of AXL Fc constructs made and tested.

The following AXL Fc constructs were made:

a. Full-length wild-type Fc fusion

b. Full-length AXL peptide 1 Fc fusion

c. AXL peptide 1 Fn(−) Fc fusion (this is the Fn− construct)

d. Full-length AXL peptide 1 Fc fusion with minor GAS6 binding site knocked out

e. AXL peptide 1 Fn(−) Fc fusion, 3× gly4ser linker between Fc and AXL

f. AXL peptide 1 Fn(−) Fc fusion, 5× gly4ser linker between Fc and AXL

g. AXL peptide 1 A72V Fn(−) Fc fusion, 3× gly4ser linker between Fc and AXL

The following Table 1 outlines the affinities of the above constructs to GAS6, with wild-type AXL as a comparison.

TABLE 1
Construct Affinities

| AXL clone | Fn domains | Fc | Linker | K_{d} (pM) |
|---|---|---|---|---|
| Wild-type Ig1 | − | None | None | 32.8 ± 0.63 |
| AXL peptide 1 Ig1 | − | None | None | 2.7 ± 0.05 |
| (a) Wild-type | + | hIgG | None | 9.2 ± 0.17 |
| (b) AXL peptide 1 | + | hIgG | None | 0.4 ± 0.01 |
| (c) AXL peptide 1 | − | hIgG | None | 2.6 ± 0.05 |
| (d) AXL peptide 1 (−) | + | hIgG | None | 2.6 ± 0.10 |
| minor site | | | | |
| (e) AXL peptide 1 | − | hIgG | 3x gly₄ser | 1.2 ± 0.03 |
| (f) AXL peptide 1 | − | hIgG | 5x gly₄ser | 1.2 ± 0.03 |
| (g) AXL peptide 1 A72V | − | hIgG | 3x gly₄ser | 0.3 ± 0.00 |

There are several conclusions that can be drawn from the data set in Table 1 above.

Fc-fusion constructs provide enhancements in affinity over the monomeric forms. For example: wild-type AXL Ig1 (monomeric) has a ˜33 pM affinity, whereas wild-type Fc fusion has an affinity of ˜9 pM and AXL peptide 1 Ig1 (monomeric) has an affinity of ˜3 pM, whereas AXL peptide 1-Fc fusion has an affinity of ˜0.4 pM

Significant affinity improvements for AXL peptide 1 over wild-type AXL. In addition, AXL peptide 1 plus the A72V mutation has a further enhancement in affinity, construct (e) compared to construct (g), over wild-type AXL.

The mechanism of increasing the affinity in the Fc fusion comes from multivalent binding to a single GAS6 molecule. Specifically, one arm of the fusion binds to the major AXL binding site while the other binds the minor. This conclusion was based on the following experimental data:
a. AXL peptide 1 Ig1 (monomer) has the same affinity as the AXL peptide 1 Fn(−) Fc fusion, (c) in the table above. This suggests that simply having two copies of AXL is insufficient for providing affinity improvement.
b. Full-length AXL peptide 1 with the minor binding site removed has the same affinity as the monomer and the Fn(−) fusion. This shows that the minor binding site has a definitive role in the affinity improvements.
c. The Fn(−) constructs are arranged such that the minor binding site is inaccessible to the large GAS6 molecule. The addition of linkers between AXL and the Fc provide additional flexibility and space, and with that a two-fold improvement in affinity is obtained. This further supports the idea that the minor binding site is important.

Overall, Example 1 shows that Fc fusions of the AXL ECD can have improved affinity to GAS6 compared to wild-type AXL. While not being bound by theory, one mechanism underlying this improved affinity is simultaneous binding of one arm of the construct to the major AXL binding site on GAS6 and the other arm to the minor AXL binding site on GAS6.

### Example 2

### Affinities of Various Axl Fc Constructs

Sequence: AXL peptide 2-Fc. The AXL peptide 2 includes amino acids 1-131 of AXL, has the Ig2 domain deleted and has both the FN domains deleted.

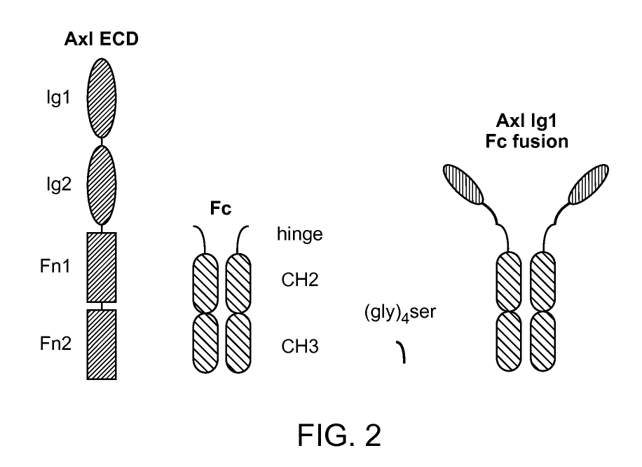

This construct includes amino acids 1-131 of AXL fused to the human IgG1, with a single Gly₄Ser linker connecting the two domains (see FIG. 2). Additionally, constructs can include various mutations in the AXL portion of the molecule, as described by the present invention. For example, embodiments can include having AXL peptide 1 Ig1 Fc fusion or AXL peptide 1 plus A72V.

The affinity of AXL peptide 1 Ig1-Fc to human GAS6 has been measured to be 1.7+/−0.03 pM.

Overall, Example 2 shows that Fc fusions of the AXL Ig1 domain can have improved affinity to GAS6 compared to wild-type AXL.

### Example 3

### An Engineered High-Affinity Axl Receptor Decoy is a Potent Inhibitor of Metastatic Disease

The Axl receptor tyrosine kinase and its activating ligand, growth arrest-specific 6 (Gash), are critical regulators of disease progression in many forms of human cancer¹⁻⁵_ENREF_1_ENREF_1. Activation of Axl has been shown to drive metastasis^{6,7} and confer therapeutic resistance⁸, resulting in advanced disease for which effective treatments have remained elusive. As a result, Axl has generated significant interest as a target for therapeutic intervention. Current approaches to antagonize Axl signaling, including antibodies^{9,10}, aptamers¹¹, small molecules¹², and receptor decoys consisting of the Axl extracellular domain^{7,13} have shown modest efficacy, but demonstrate the therapeutic value of targeting this signaling pathway. The present example describes engineered soluble fragments of Axl (sAxl) with improved binding affinity to Gas6, which in turn afforded enhanced antagonistic properties. These sAxl variants bound Gas6 with apparent affinities as low as 93 fM, representing one of the strongest protein-protein interactions ever reported, and a 350-fold improvement over the wild-type interaction. Crystal structures of Axl variants in complex with Gas6 reveal how a small number of Axl mutations stabilize the bound conformation of its ligand, driving the affinity improvement. The engineered Axl variants systemically sequester Gas6 when administered in vivo, and inhibit disease progression in aggressive metastatic models of human breast, ovarian, and pancreatic cancers, with efficacy far exceeding that of wild-type sAxl. Our results demonstrate the value of targeting Gas6 to treat metastatic disease and highlight the important role that affinity plays in driving the efficacy of a receptor decoy.

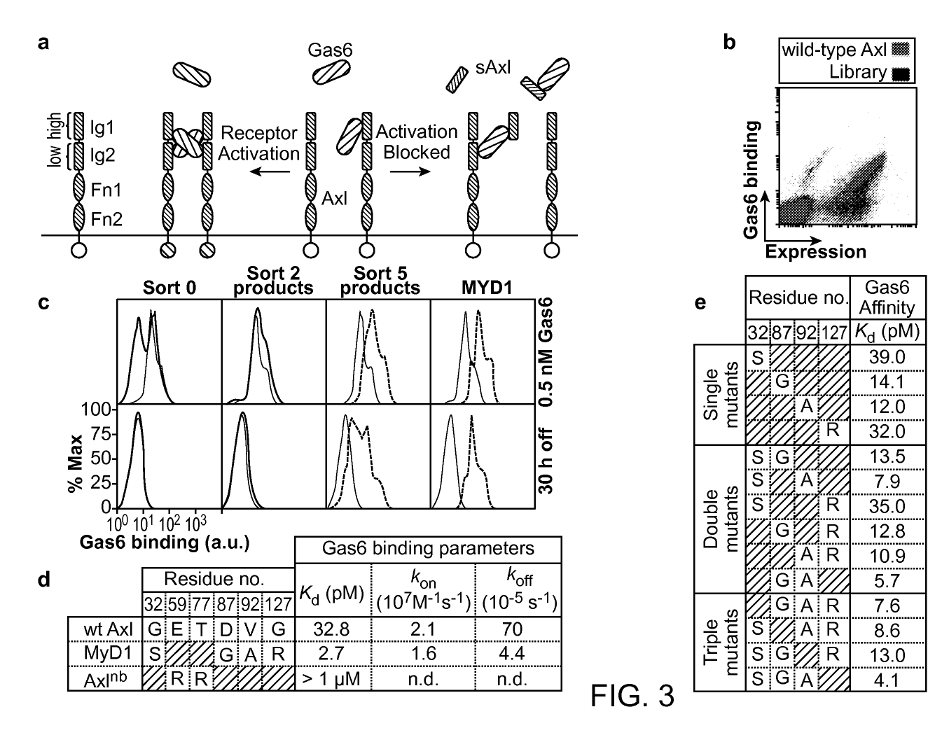

Soluble fragments of native receptors that bind to and neutralize their cognate ligands can be effective therapeutics¹⁴, illustrated by the clinical success of Enbrel (etanercept) and Eylea (aflibercept). Soluble forms of the Axl extracellular domain (sAxl)^{7,13} have been explored as anti-metastatic agents that function to block the activity of its sole ligand^{15,16}, growth arrest-specific 6 (Gas6) (FIG. 3a). Exploiting the native interaction has distinct advantages: off-target activity is eliminated as sAxl only binds Gas6, and Gas6-induced signaling through Mer, an Axl family member also linked to metastasis^{17,18}, will be similarly disrupted. While wild-type sAxl has shown only modest anti-tumor efficacy, it has further validated Axl as a therapeutic target in metastatic disease. This example demonstrates that increased therapeutic efficacy can be achieved with higher affinity sAxl variants that more effectively compete with endogenous Axl for Gas6 binding. This example describes engineered sAxl variants with improved affinity to Gas6 and demonstrates their ability to inhibit metastasis in vivo.

Axl interacts with Gas6 through two independent epitopes: a high-affinity major binding site on the first immunoglobulin-like domain (Ig1) and a low-affinity minor site on Ig2 (FIG. 3a)¹⁹. The major binding site drives the receptor's overall affinity for Gas6 by leveraging an unusual geometry in which anti-parallel β-strands from Gas6 and Axl align on edge, forming a continuous β-sheet that bridges the interface¹⁹. This architecture creates an elongated binding epitope on Axl, involving residues throughout the Ig1 domain. The importance of the major binding site, and the extended nature of its interface, prompted us to design an in vitro evolution strategy where mutations were randomly introduced into the Axl Ig1 domain through error-prone PCR.

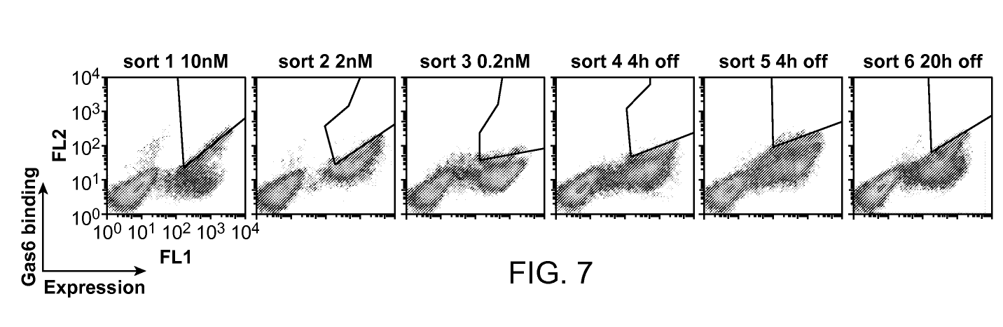

The resulting Axl Ig1 library was displayed as fusion proteins on the yeast cell surface where a subset retained binding to Gas6 (FIG. 3b)^{20,21}_ENREF_20. Fluorescence-activated cell sorting (FACS) was used to isolate clones with high affinity for Gas6 (for details see Methods, FIG. 7), and after six rounds of screening only three unique clones remained. The variant MYD1 showed the most significant improvement over wild-type Axl Ig1 in both amount of Gas6 bound and persistent Gas6 binding (FIG. 3c), and was chosen for further analysis.

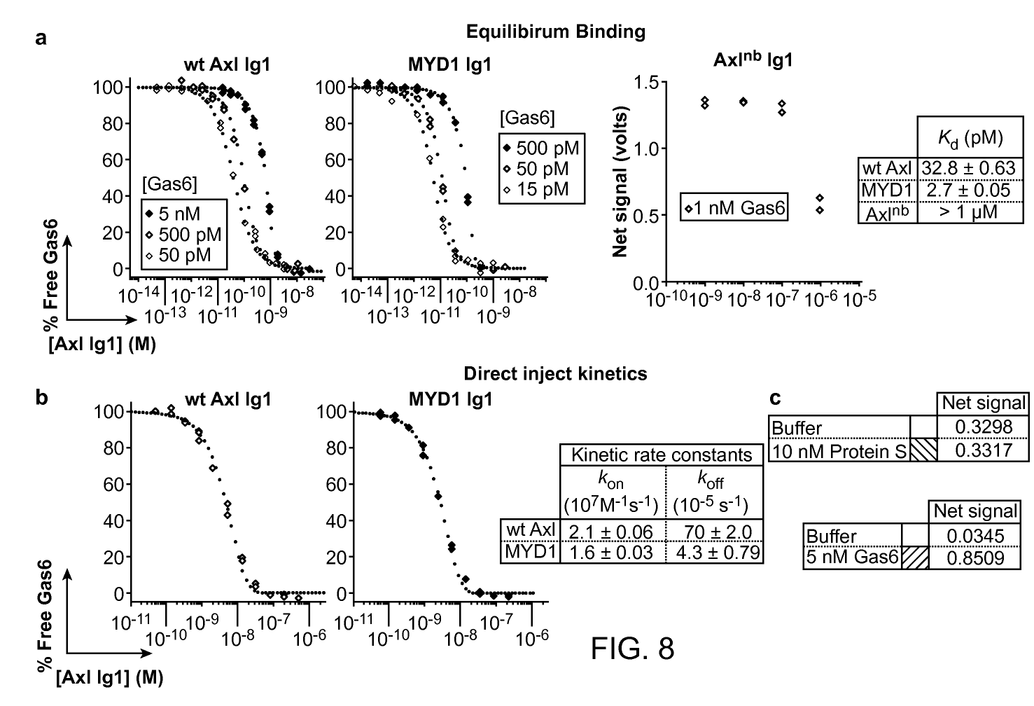

To characterize the affinity of recombinantly produced MYD1 the kinetic exclusion assay (KinExA)²² was used, a method uniquely suited to accurately measure binding interactions with affinities ranging from nanomolar to femtomolar. For comparison, wild-type Axl Ig1, and ‘Axl^{nb}’, a non-binding variant containing two previously identified deleterious mutations (E59R/T77R)¹⁹ that abolish binding to Gas6 were also evaluated. Wild-type Axl Ig1 bound Gas6 with an equilibrium dissociation constant (K_{d}) of 32.8 pM, and was characterized by a fast association rate (kₒₙ) of 2.1×10⁷ M⁻¹s⁻¹ (FIG. 3d and FIG. 8). This exceptionally high affinity offers a plausible explanation for why previous anti-Axl therapeutic candidates, which possess affinities to human Axl in the nM range, have demonstrated limited efficacy^{7,9,11,12}. MYD1 had an affinity of 2.7 pM, a 12-fold increase over wild-type that was predominantly driven by a slower dissociation rate (k_{off}). As expected, Axl^{nb} showed minimal binding to Gas6 at concentrations up to 1 μM. Importantly, MYD1 retains the specificity of wild-type Axl, showing no binding to protein S (FIG. 8), a homolog of Gas6 that is a critical regulator of the coagulation cascade^{23,24}.

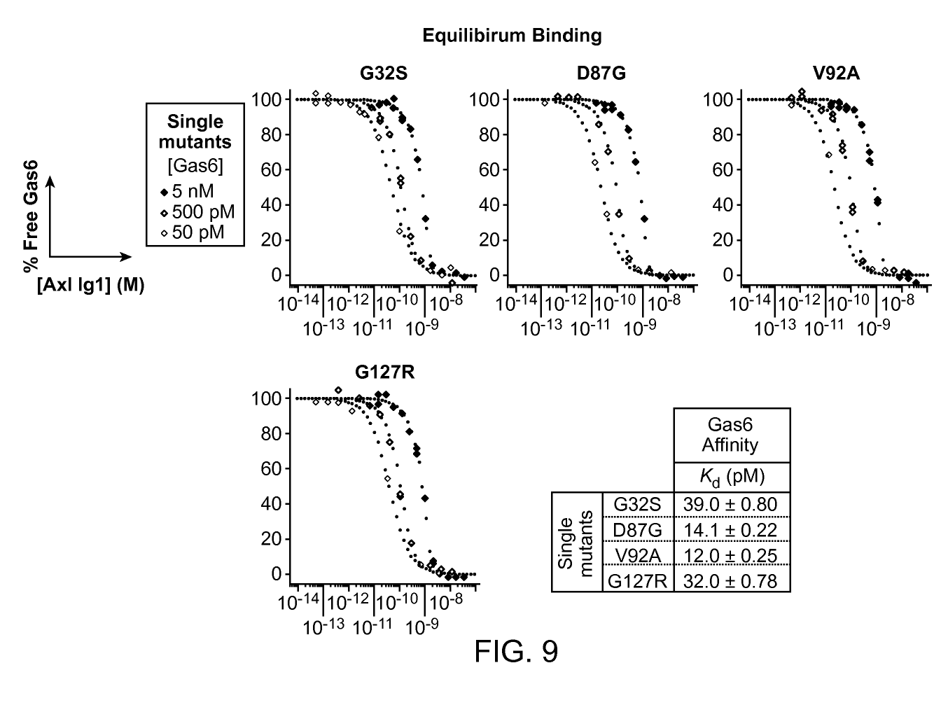

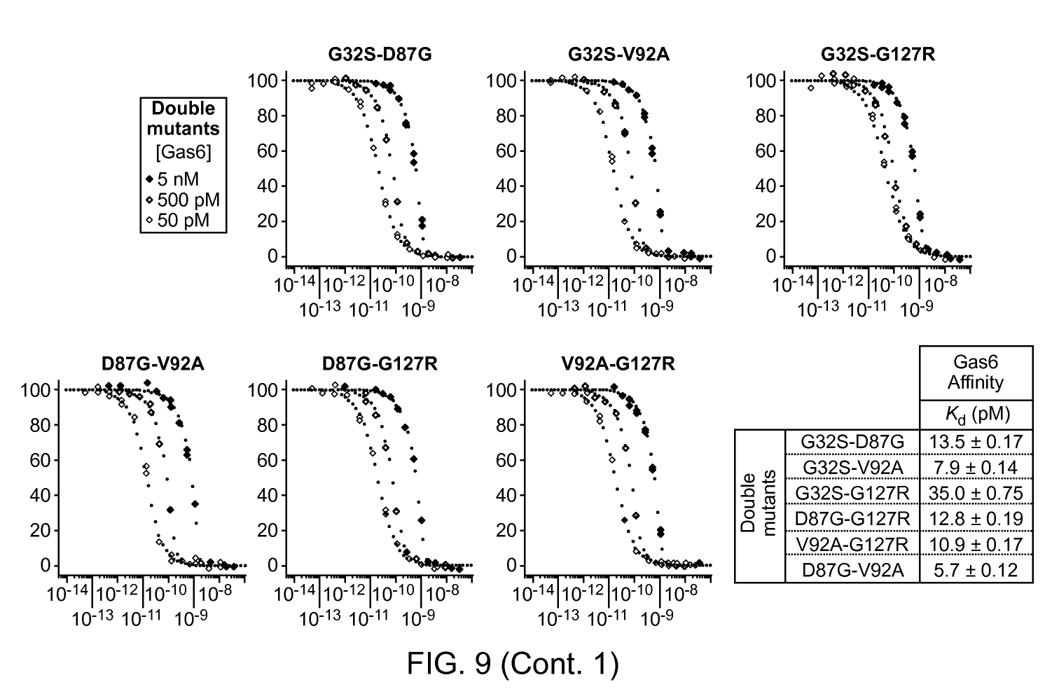

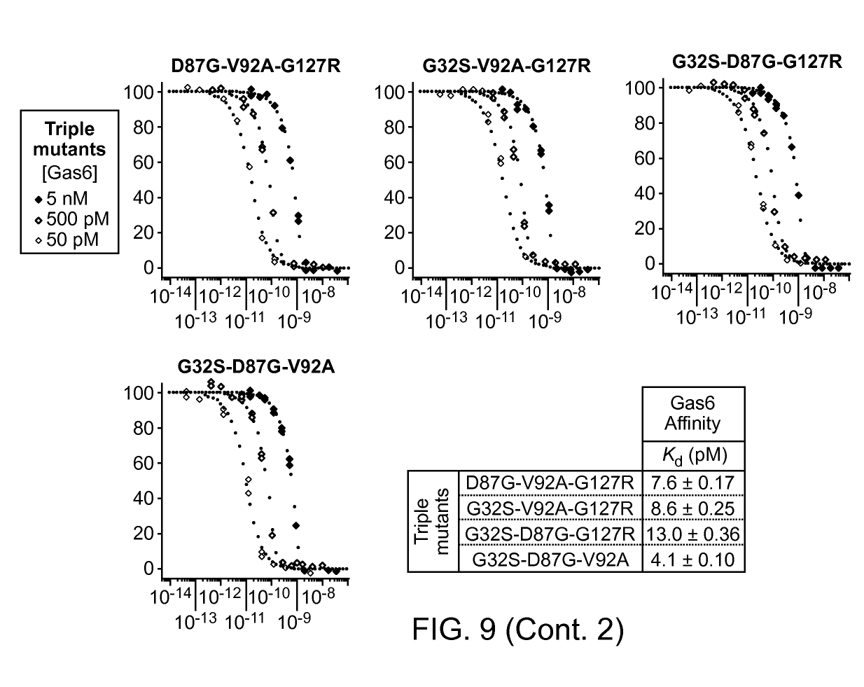

To investigate how the four mutations in MYD1 affect its affinity for Gas6, every permutation of single, double, and triple mutants were recombinantly expressed and analyzed. KinExA studies indicated that a majority of MYD1's increased affinity was due to two independent mutations, D87G and V92A (FIG. 3e and FIG. 9). The remainder derives from a synergistic interaction between G32S, a slightly deleterious mutation on its own, and V92A. Effects of the single point mutations on protein stability were also explored. Thermal melts showed similar stabilities between wild-type Axl and MYD1, though significant offsetting differences existed between the single mutants (Table 2), suggesting G127R was selected for its stabilizing effects.

TABLE 2
Thermal stabilities of Axl Ig1 variants

| | Residue no. | | | | Thermal stability |
|---|---|---|---|---|---|
| | 32 | 87 | 92 | 127 | Tₘ (° C.) |
| wt Axl | G | D | V | G | 53 ± 0.6 |
| MYD1 | S | G | A | R | 54 ± 0.9 |
| Single | S | | | | 52 ± 0.6 |
| mutants | | G | | | 55 ± 0.4 |
| | | | A | | 47 ± 1.0 |
| | | | | R | 57 ± 0.4 |

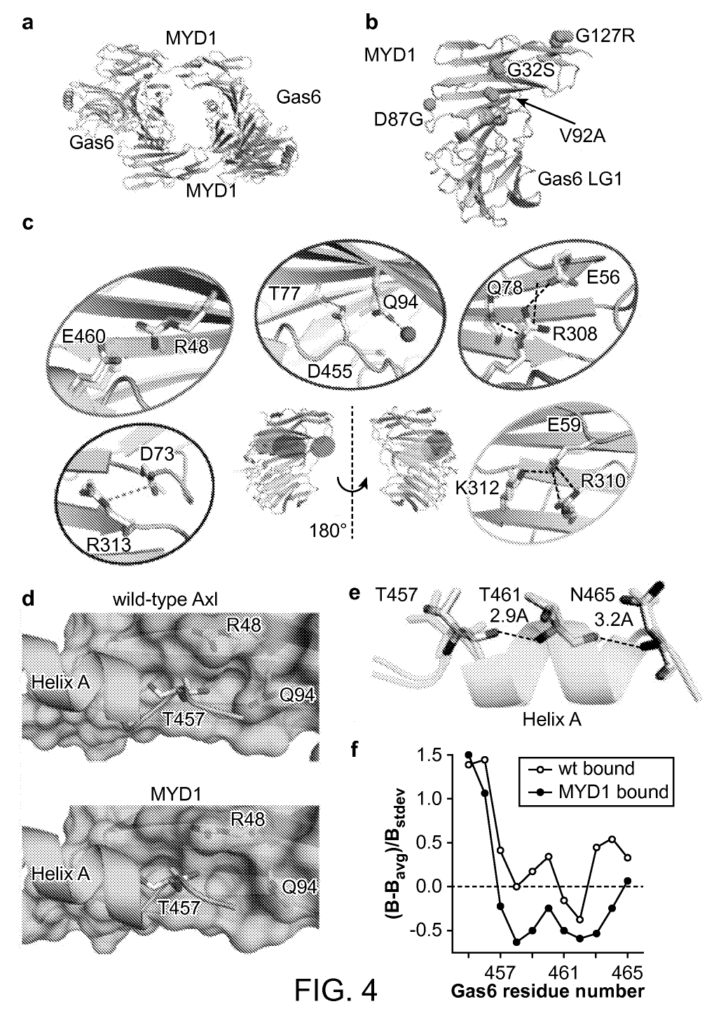

To gain insights into the structural basis for the affinity increase, the Gas6/MYD1 co-complex was crystallized. The resulting structure maintained the overall geometry and 2:2 stoichiometry of the wild-type Gas6/Axl complex¹⁹ (r.m.s.d. 1.9 Å, FIG. 4a and Table 3). Analysis revealed that affinity improvements were a result of two distinct structural mechanisms: the formation of additional contacts with Gas6, and a MYD1-induced conformational change on Gas6 that stabilizes the bound structure of the ligand.

TABLE 3
Crystallographic Statistics

| | Gas6/MYD1 | Gas6/MYD1-72 |
|---|---|---|
| Crystallographic parameters | | |
| Space group | P3₂21 | P3₂21 |
| Unit-cell paramters | | |
| a, b, c (Å) | 112.37, 112.37, | 113.04, 113.04, |
| | 361.26 | 361.86 |
| α, β, γ (°) | 90, 90, 120 | 90, 90, 120 |
| Data collection statistics | | |
| Wavelength (Å) | 1.03 | 0.98 |
| Resolution limits (Å) | 38.01-3.07 | 38.15-3.50 |
| Number of observed reflections | 601747 | 244258 |
| Number of unique reflections | 50592 | 33726 |
| Completeness (%) | | |
| overall/outer shell | 99.9/100 | 96.6/91.0 |
| Redundancy | | |
| overall/outer shell | 11.9/7.1 | 7.2/3.4 |
| R_{sym}^{a} (%) | | |
| overall/outer shell | 11.8/100.0 | 13.6/74.4 |
| I/σ | | |
| overall/outer shell | 20.4/2.1 | 11.1/1.6 |
| Refinement statistics | | |
| Resolution limits (Å) | 38.01-3.07 | 38.15-3.50 |
| Number of reflections (%) | 47994 (99.9) | 31985 (96.6) |
| Reflections used for R_{free} | 2526 | 1684 |
| R_{(working)}^{b} (%) | 20.2 | 20.3 |
| R_{free} (%) | 24.3 | 24.5 |
| Model contents/average B (Å)² | | |
| Protein atoms (includes | 8901/92.5 | 8905/114.7 |
| sugars) | | |
| Ions | 15/106.5 | 14/141.2 |
| Water molecules | 14/61.1 | 3/76.2 |
| Ramachandran plot (%) | | |
| favored/outliers | 93.19/0.97 | 93.28/1.15 |
| RMS deviations | | |
| Bond length (Å) | 0.007 | 0.007 |
| Bond angle (°) | 1.279 | 1.247 |

^{a}R_{sym} = Σ|lavg − li|/Σli
^{b}R_{factor} = Σ|Fp − Fpcalc|/ΣFp, where Fp and Fpcalc are observed and calculated structure factors; R_{free} is calculated within 5% of the data.

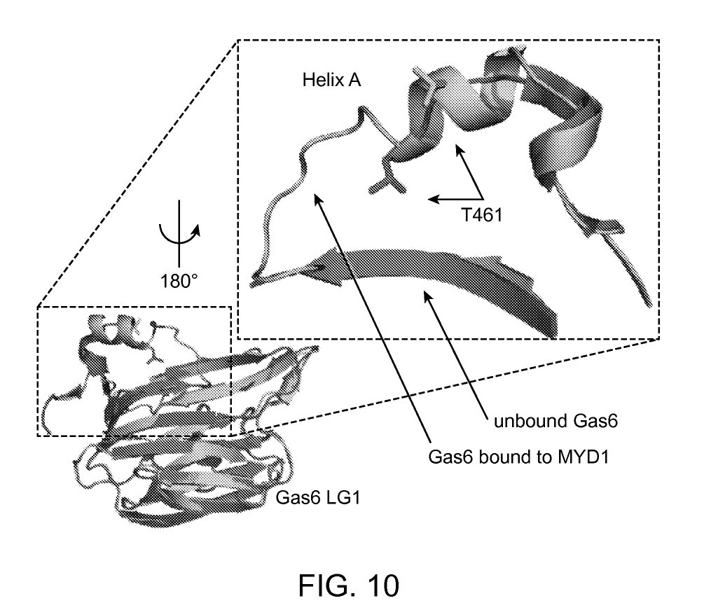

Of the four mutations in MYD1, only V92A lied within the binding interface (FIG. 4b); however, side chains throughout the interface underwent significant reorganization resulting in a net gain of three new contacts with Gas6 with respect to the wild-type structure (FIG. 4c and Table 4). The valine to alanine mutation at position 92 alleviated steric crowding and permitted local structural reorganization, resulting in the formation of an elongated groove on Axl at the binding interface (FIG. 4d). This groove in turn induced a dramatic structural change across the interface on Gas6. As previously reported, a loop on Gas6 LG1 is disordered when unbound²⁵, and association with Axl resulted in the formation of a short α-helix (FIG. 10)¹⁹. In the wild-type complex, this helix (Helix A) was held together by a single backbone hydrogen bond between T461^{Gas6} and N465^{Gas6} and made minimal contact with Axl¹⁹. In the MYD1 complex, two distinct but complimentary interactions acted to stabilize Helix A. Adjacent to position 92, the R48^{MYD1} side chain rotated, forming a more favorable interaction with E460^{Gas6} (FIG. 4c, red oval). More importantly, the groove created by V92A permits T457^{Gas6}, the i-4 residue with respect to T461^{Gas6}, to reorient and resulted in the formation of new backbone hydrogen bond with T461^{Gas6} (FIG. 4e). This hydrogen bond was consistent with the commonly observed mechanism of stabilizing helices through N-terminal capping²⁶. Together, those changes stabilized the bound conformation of Gas6, as reflected by the lower crystallographic B-factors in Helix A relative to the rest of the MYD1 structure, compared to wild-type Axl (FIG. 4f, see Methods for details). The Axl-induced conformational change that occurred within the bound Gas6 ligand was a unique mechanism of affinity enhancement, as the engineered interaction achieved improved shape complementarity due to restructuring of the modified binding partner.

TABLE 4
Comparison of intermolecular contacts in
wild-type Axl and Axl MYD1 co-complexes
Gas6/wild-type Axl

| | | | | |
|---|---|---|---|---|
| Res | wt Axl | Res | Gas6 | Distance (Å) |

Hydrogen bonds†

| | | | | |
|---|---|---|---|---|
| 48 | ARG [NH1] | 456 | THR [O] | 3.47 |
| 56 | GLU [OE2] | 308 | ARG [NH1] | 3.32 |
| 73 | ASP [OD1] | 314 | LUE [N] | 2.54 |
| 74 | SER [N] | 312 | LYS [O] | 2.91 |
| 74 | SER [O] | 313 | ARG [NH2] | 3.15 |
| 74 | SER [O] | 312 | LYS [N] | 3.02 |
| 75 | THR [OG1] | 313 | ARG [NH2] | 2.51 |
| 76 | GLN [N] | 310 | ARG [O] | 3.10 |
| 76 | GLN [O] | 310 | ARG [N] | 2.94 |
| 78 | GLN [N] | 308 | ARG [O] | 2.74 |
| 78 | GLN [O] | 308 | ARG [N] | 2.93 |
| 80 | PRO [O] | 299 | ARG [NH2] | 2.29 |
| 83 | GLU [O] | 302 | SER [OG] | 2.59 |

Electrostatic interactions

| | | | | |
|---|---|---|---|---|
| 48 | ARG [NH1] | 460 | GLU [OE1] | 3.57 |
| 48 | ARG [NH2] | 460 | GLU [OE1] | 2.77 |
| 56 | GLU [OE2] | 308 | ARG [NH1] | 3.32 |
| 56 | GLU [OE2] | 308 | ARG [NH2] | 3.67 |
| 59 | GLU [OE1] | 310 | ARG [NH2] | 3.66 |
| 59 | GLU [OE2] | 310 | ARG [NH1] | 3.30 |
| 59 | GLU [OE2] | 310 | ARG [NE] | 3.81 |
| 59 | GLU [OE2] | 310 | ARG [NH2] | 2.98 |
| 59 | GLU [OE2] | 312 | LYS [NZ] | 3.38 |
| 73 | ASP [OD2] | 313 | ARG [NE] | 3.98 |

Gas6/MYD1

| | | | | |
|---|---|---|---|---|
| Res | MYD1 | Res | Gas6 | Distance (Å) |

Hydrogen bonds

| | | | | |
|---|---|---|---|---|
| 48 | ARG [NH1] | 456 | THR [O] | 3.21 |
| 56 | GLU [OE2] | 308 | ARG [NH2] | 2.61 |
| 59 | GLU [OE2] | 312 | LYS [NZ] | 2.57 |
| 73 | ASP [OD1] | 314 | LEU [N] | 2.79 |
| 74 | SER [N] | 312 | LYS [O] | 2.98 |
| 74 | SER [O] | 313 | ARG [NH2] | 2.90 |
| 74 | SER [O] | 312 | LYS [N] | 3.03 |
| 75 | THR [OG1] | 313 | ARG [NH2] | 2.68 |
| 76 | GLN [N] | 310 | ARG [O] | 2.91 |
| 76 | GLN [O] | 310 | ARG [N] | 2.90 |
| 77 | THR [OG1] | 455 | ASP [OD2] | 3.08 |
| 78 | GLN [N] | 308 | ARG [O] | 2.73 |
| 78 | GLN [O] | 308 | ARG [N] | 2.92 |
| 78 | GLN [OE1] | 308 | ARG [NH2] | 2.91 |
| 80 | PRO [O] | 299 | ARG [NH2] | 2.35 |
| 83 | GLU [O] | 302 | SER [OG] | 2.80 |

Electrostatic interactions

| | | | | |
|---|---|---|---|---|
| 48 | ARG [NH1] | 460 | GLU [OE1] | 3.85 |
| 48 | ARG [NH1] | 460 | GLU [OE2] | 3.27 |
| 48 | ARG [NH2] | 460 | GLU [OE1] | 3.47 |
| 56 | GLU [OE2] | 308 | ARG [NH1] | 3.62 |
| 56 | GLU [OE2] | 308 | ARG [NH2] | 3.06 |
| 59 | GLU [OE2] | 310 | ARG [NE] | 3.84 |
| 59 | GLU [OE2] | 310 | ARG [NH1] | 2.88 |
| 59 | GLU [OE2] | 310 | ARG [NH2] | 3.37 |
| 59 | GLU [OE2] | 312 | LYS [NZ] | 2.79 |
| 59 | GLU [OE1] | 310 | ARG [NH1] | 3.73 |

†Contacts common to both structures are grayed out in both lists.

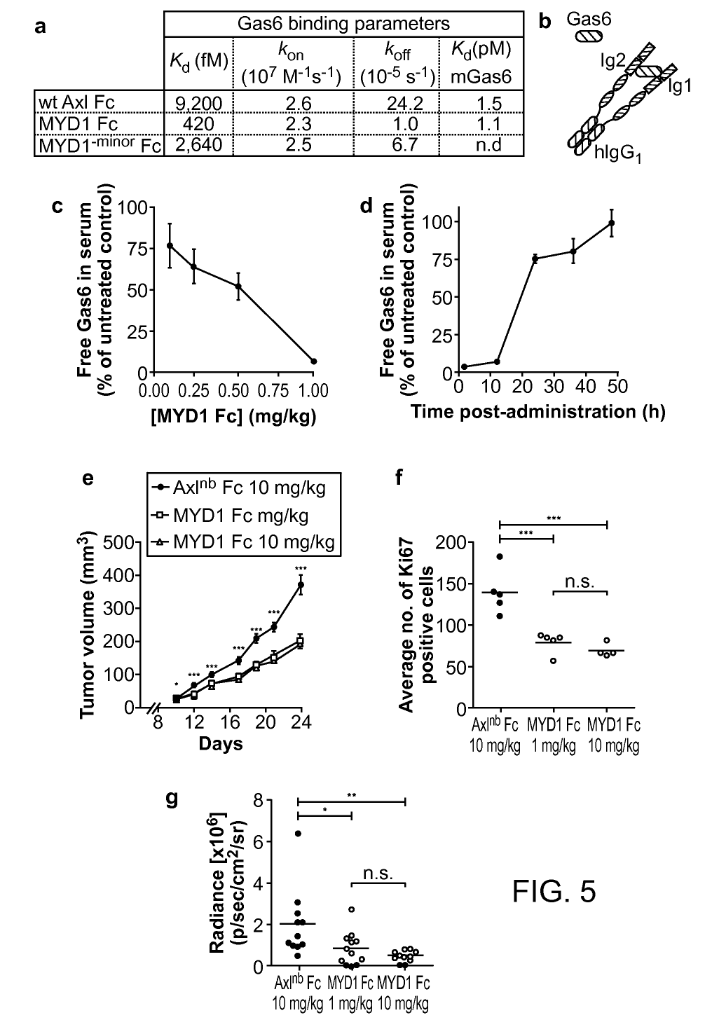

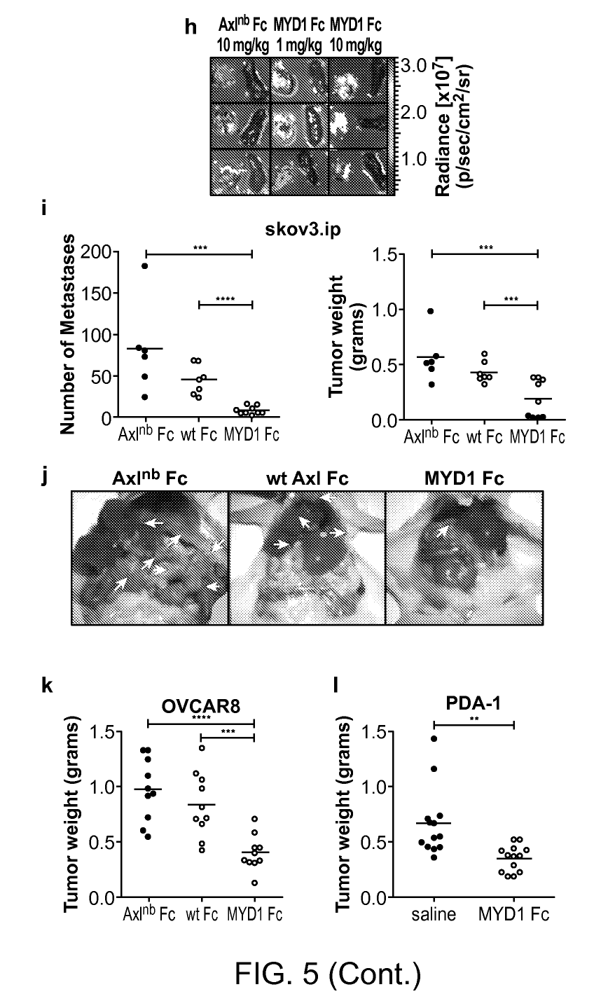

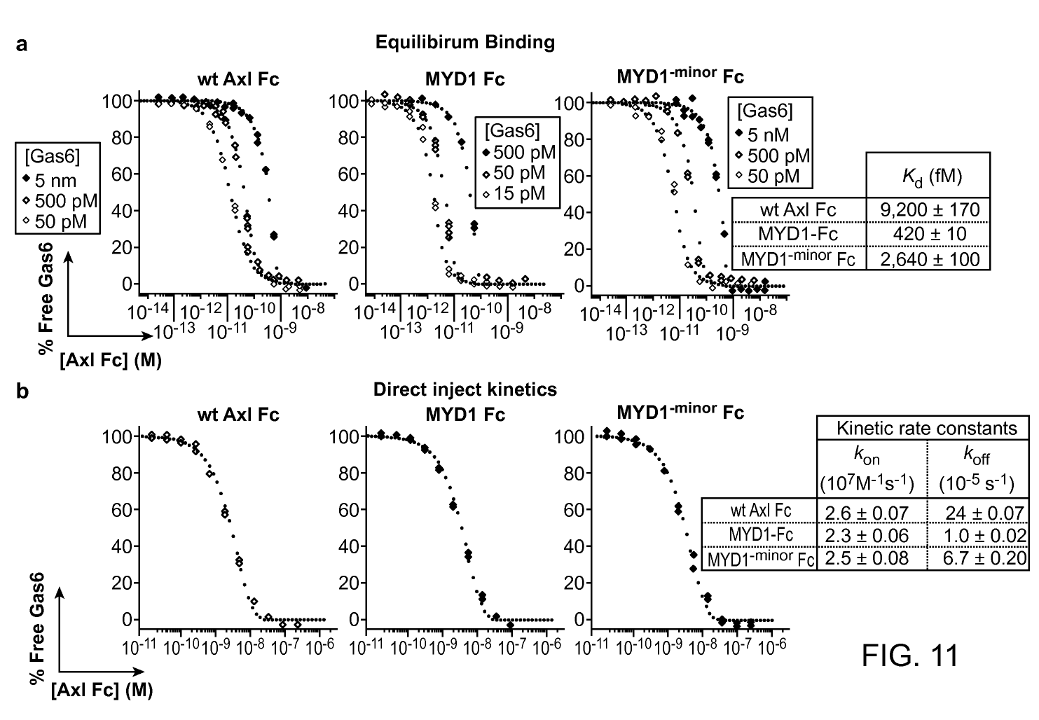

In order to characterize the in vivo activity of MYD1, the wild-type Axl, Axl^{nb} and MYD1 Ig1 fragments were cloned back into the full-length Axl receptor (FIG. 3a) and then fused to the Fc domain of a human IgG₁. Significant improvements in the apparent affinities of the Fc fusions were observed relative to the single domain Ig1 constructs (FIG. 5a and FIG. 11), with MYD1 Fc having an affinity of 420 fM for human Gas6, an 80-fold increase over wild-type Axl Ig1. Both wild-type Axl Fc and MYD1 Fc bound mouse Gas6 with a K_{d} of ˜1 pM (FIG. 5a), as the library was screened against human Gas6. To evaluate the role of the reintroduced Gas6 minor binding site, an additional fusion (MYD1^{-minor} Fc) was generated that was identical to MYD1 Fc except for two mutations (K204E/T208E), which eliminated this site on Axl Ig2. MYD1^{-minor} Fc had the same affinity for Gas6 as the monovalent MYD1 Ig1 (FIG. 5a), illustrating that full-length Fc fusions require a functional minor site for increased binding. Based on these results, a binding mechanism was proposed in which a single molecule of Gas6 interacts with both copies of Axl in the Fc fusion; one Axl binds the major site, while the other binds the minor site (FIG. 5b). This created a significant avidity effect that increased the apparent affinity of the overall interaction.

Next, whether MYD1 Fc could effectively sequester Gas6 in the blood was tested. Non-tumor bearing nude mice were given single doses of MYD1 Fc ranging from 0.1-1 mg/kg, and the amount of free Gas6 was determined from serum samples obtained after 12 hours (FIG. 5c). At 1 mg/kg a negligible amount of free Gas6 remained, and a linear dose response was observed with decreasing concentrations. A time course in which mice received a single 1 mg/kg dose revealed that MYD1 Fc rapidly sequestered all Gas6 in the blood and kept free Gas6 levels suppressed for over 36 hours (FIG. 5d). Together, these studies show that MYD1 Fc is highly active in vivo, where it completely eliminated free Gas6 systemically upon administration.

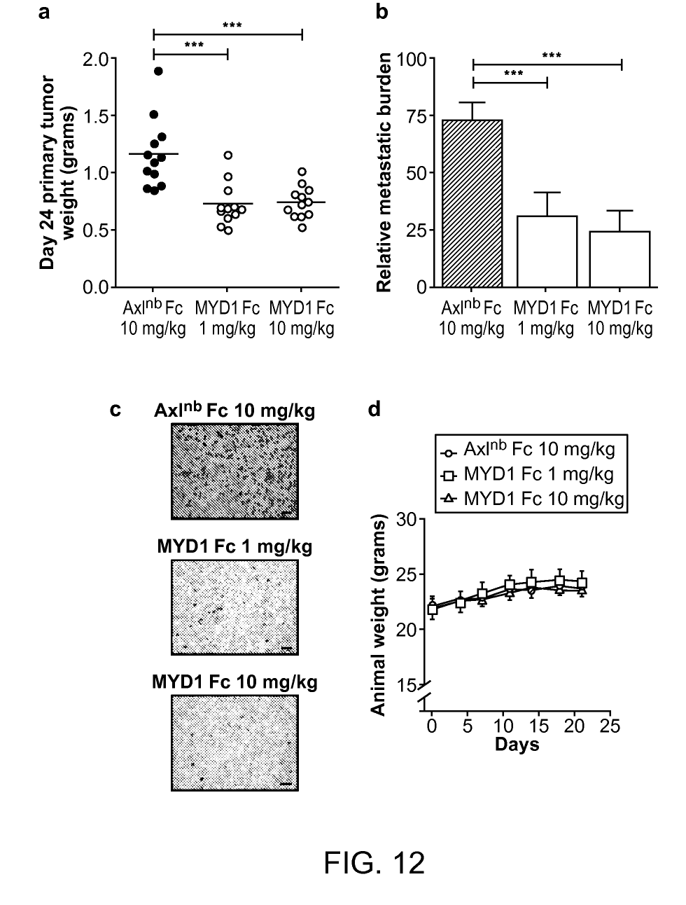

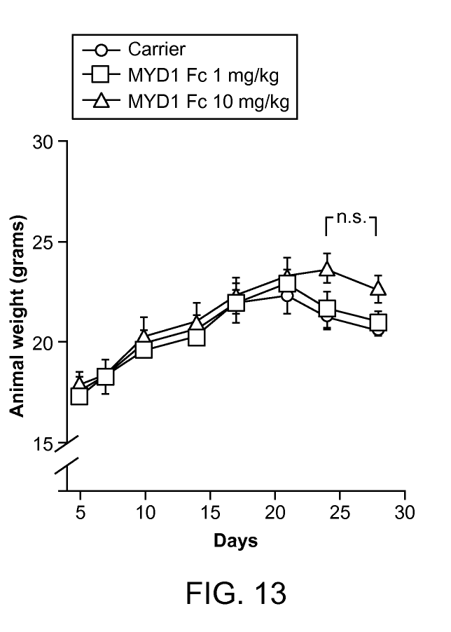

To test whether Gas6 sequestration would disrupt tumor progression in metastatic models of human cancer, MYD1 Fc was tested in a 4T1 orthotopic breast cancer model. In this model, tumors in the mammary fat pad aggressively metastasize to the lungs. Mice were implanted with 4T1 cells and randomized into three treatment groups: Axl^{nb} Fc at 10 mg/kg, and MYD1 Fc at 1 or 10 mg/kg. At both doses, MYD1 Fc was able to significantly reduce the size of primary tumors compared to the Axl^{nb} Fc controls, and inhibited proliferation, as detected by the marker Ki67 (FIG. 5e, f and FIG. 12). At the conclusion of the study, mice in the high and low dose MYD1 Fc groups had on average 60% and 80% fewer lung metastases, respectively, compared to control animals (FIG. 5g, h). MYD1 Fc showed no observable signs of gross toxicity and animal weight across groups remained consistent (FIG. 12). A full toxicology study in which non-tumor bearing animals were given repeat doses of MYD1 Fc revealed no adverse events or signs of toxicity (FIG. 13, Table 5).

TABLE 5
Toxicology study results including complete blood counts and chemistry panel

| | | MYD1 Fc | | | |
|---|---|---|---|---|---|
| | Carrier | 1 mg/kg | 10 mg/kg | Normal | Units |
| Haematological | | | | | |
| HCT | 47 ± 0.7 | 38 ± 10.3 | 46 ± 0.8 | 39.0-47.0 | % |
| RBC | 9 ± 0.1 | 9 ± 0.0 | 8 ± 0.0 | 7.0-8.8 | M/μl |
| HGB | 15 ± 0.2 | 14 ± 0.1 | 14 ± 0.2 | 13.7-16.4 | gm/dL |
| WBC | 7 ± 1.0 | 4 ± 0.8 | 5 ± 1.9 | 5.5-9.3 | k/μl |
| Platelet (estimate) | Adequate | Adequate | Adequate | Adequate | |
| Chemistry Panel | | | | | |
| Cholesterol | 81 ± 1.5 | 28 ± 25.3 | 74 ± 1.8 | N/A | mg/dL |
| Glucose | 194 ± 3.5 | 200 ± 8.4 | 198 ± 22.6 | 184-220 | mg/dL |
| Calcium | 11 ± 0.1 | 11 ± 1.1 | 11 ± 0.1 | 8.9-9.7 | mg/dL |
| Phosphorous | 13 ± 0.6 | 12 ± 1.5 | 13 ± 1.1 | N/A | mg/dL |
| Carbon dioxide | 26 ± 1.1 | 22 ± 0.4 | 23 ± 2.2 | N/A | mmol/L |
| Chloride | 112 ± 2.6 | 124 ± 0.3 | 122 ± 2.7 | N/A | mmol/L |
| Potassium | 8 ± 0.4 | 8 ± 0.5 | 8 ± 0.9 | 3.0-9.6 | mmol/L |
| Sodium | 152 ± 4.6 | 156 ± 0.3 | 154 ± 2.1 | 114-154 | mmol/L |
| Hepatic | | | | | |
| ALK | 70 ± 17.9 | 83 ± 11.2 | 71 ± 15.5 | 76-160 | U/L |
| AST | 97 ± 18.3 | 136 ± 25.2 | 107 ± 20.4 | 192-388 | U/L |
| ALP | 106 ± 3.8 | 72 ± 34.7 | 79 ± 33.0 | 171-183 | IU/L |
| Albumin | 3.6 ± 0.0 | 3.3 ± 0.5 | 3.3 ± 0.5 | 3.2-3.6 | g/dL |
| Protein (total) | 6.1 ± 0.1 | 6.7 ± 0.3 | 6.4 ± 0.2 | 5.0-6.2 | g/dL |
| Bilirubin (total) | 0.1 ± 0.1 | 0.1 ± 0.1 | 0.1 ± 0.0 | N/A | mg/dL |
| GGT | 3 ± 2.1 | 1.7 ± 0.9 | 1.7 ± 0.3 | N/A | U/L |
| Globulin | 2.5 ± 0.1 | 3.3 ± 0.8 | 3.2 ± 0.4 | N/A | |
| Renal | | | | | |
| BUN | 23 ± 0.6 | 27 ± 0.9 | 23 ± 0.7 | 20.3-24.7 | mg/dL |
| Creatinine | 0.5 ± 0.3 | 0.5 ± 0.2 | 0.7 ± 0.0 | 0.1-1.1 | mg/dL |

Several forms of cancer, including ovarian and pancreatic, pose a major clinical challenge as patients usually present with significant metastatic disease at the time of diagnosis²⁷. Therefore the therapeutic efficacy of MYD1 Fc on established disease was evaluated. Two ovarian cancer models were chosen based on their ability to recapitulate the human condition, in particular diffuse metastatic disease throughout the peritoneal cavity. In the first study, human skov3.ip cells were injected intraperitoneally and allowed to establish metastases for one week prior to treatment. Mice were then randomized into three treatment groups: Axl^{nb} Fc, wild-type Axl Fc, or MYD1 Fc, all at 10 mg/kg. At the conclusion of the study, mice treated with MYD1 Fc had over 90% fewer abdominal metastases and nearly a 70% reduction in total weight of tumor tissue compared to Axl^{nb} controls (FIG. 5i, j). This efficacy far exceeded that obtained by wild-type Axl Fc, which showed only moderate improvement over the Axl^{nb} Fc controls.

Next MYD1 Fc was tested in a model of well-established disease, where the amount of preexisting metastases was substantially increased by implanting five times as many human OVCAR8 cells and waiting two weeks prior to treatment. In this model, MYD1 Fc again showed significant therapeutic efficacy, reducing the overall weight of tumor tissue over 60% compared to Axl^{nb} controls (FIG. 5k). As in the skov3.ip model, mice treated with wild-type Axl Fc showed only modest improvements compared to the Axl^{nb} Fc group. MYD1 Fc was also highly efficacious in a pancreatic cancer model, reducing metastatic disease by nearly 50% compared to saline controls (FIG. 5l).

These studies show that MYD1 Fc is a potent inhibitor of disease progression in vivo, even in aggressive models of well-established, preexisting disease. These data validated the strong role that the Gas6/Axl signaling axis plays in disease progression, and demonstrated that systemically inhibiting Gas6 is an attractive strategy for treating metastatic disease. These results also highlighted an important correlation between increased binding affinity and improved therapeutic efficacy in this system, as the difference between Axl^{nb} Fc, wild-type Axl Fc, and MYD1 Fc is their affinities for Gas6.

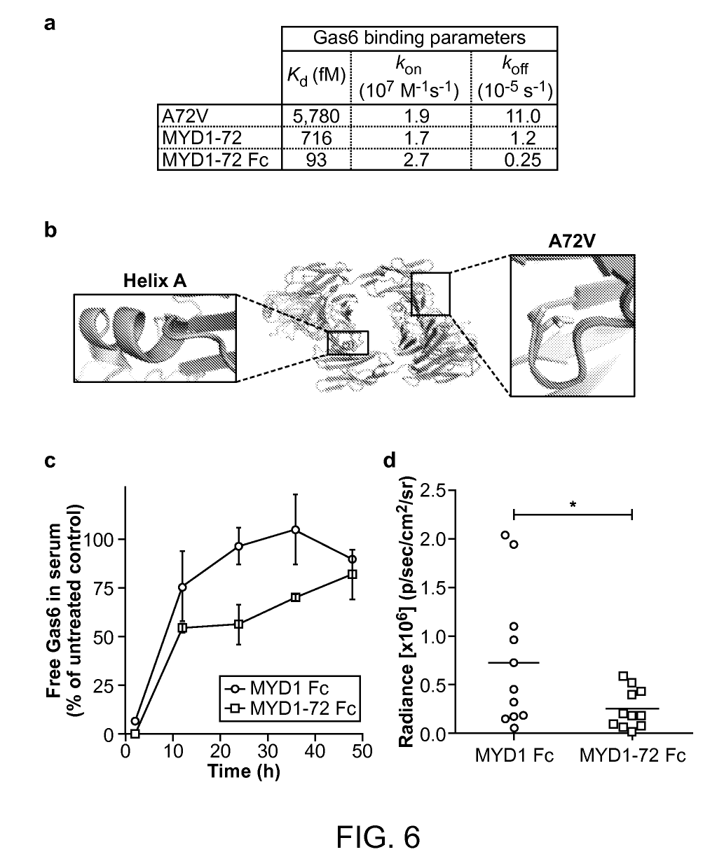

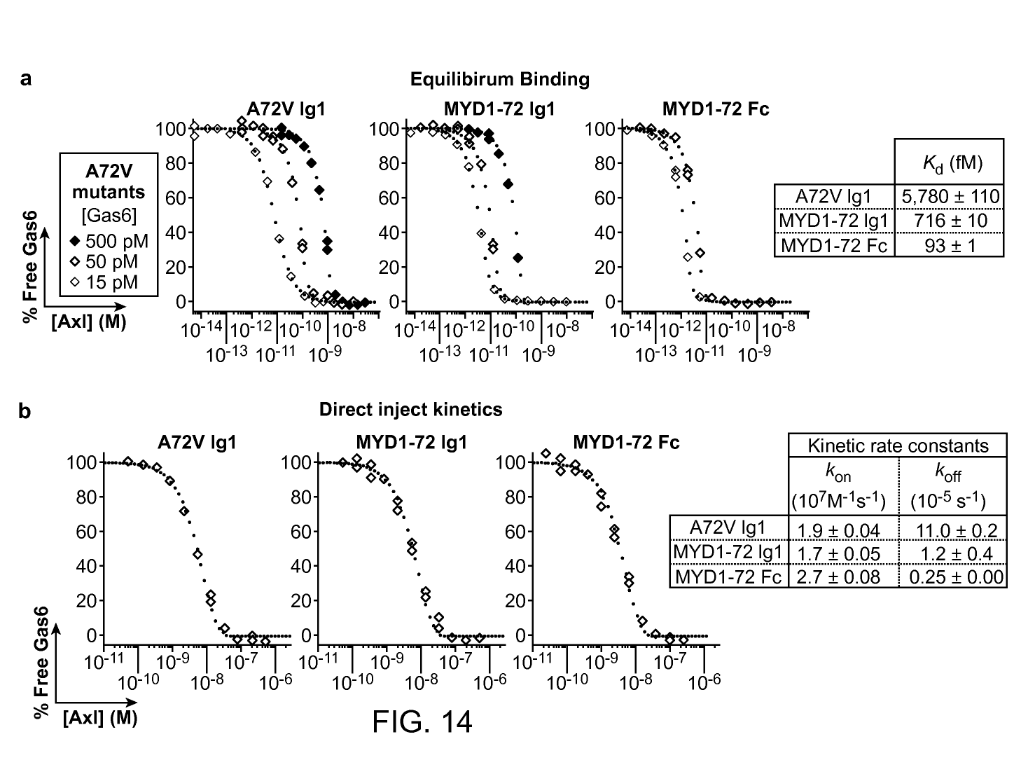

To further explore the relationship between affinity and efficacy, sequence analysis was performed on intermediate library sorts to identify additional gain-of-function mutations. Analysis of 141 randomly sampled clones resulted in twenty-five unique variants and revealed three commonly occurring mutations: A72V, D87G, and V92A, of which the latter two are contained in MYD1 (Table 6). The high frequency of A72V, which was also present multiple times as a single mutation, suggested it was beneficial. Axl Ig1 A72V was recombinantly produced and its affinity was determined to be 5.8 pM (FIG. 6a and FIG. 14), only two-fold weaker than MYD1. In the Gas6/MYD1 structure, residue 72 is located away from the other four mutations, and none of the beneficial structural changes noted in the complex (FIG. 4c) occurred in the region of position 72. Combining MYD1 and A72V resulted in an Axl variant (MYD1-72) with an affinity for Gas6 of 716 fM, 45-fold higher than wild-type Axl Ig1 (FIG. 6a and FIG. 14). Next, MYD1-72 was produced as a full-length Fc fusion (MYD1-72 Fc), which further improved its apparent Gas6 binding affinity to 93 fM (FIG. 6a and FIG. 14). This remarkable affinity, which is over 350-fold tighter than wild-type Axl Ig1, represented one of the tightest engineered or natural protein-protein interactions reported to-date.

TABLE 6
Sequence analysis of Axl Ig1 variants isolated through library screening
Axl Ig1 variants (141 total random clones sequenced, 25 unique variants)

| Residue # | 19 | 23 | 26 | 27 | 32 | 33 | 38 | 44 | 61 | 65 | 72 | 74 | 78 | 79 | 86 | 87 | 88 |
|---|---|---|---|---|---|---|---|---|---|---|---|---|---|---|---|---|---|
| wt Axl | A | T | E | E | G | N | T | T | H | D | A | S | Q | V | Q | D | D |
| MYD1 | | | | | S | | | | | | | | | | | G | |
| MYD2 | | | G | | | | | | | | | | | M | | | |
| MYD3 | | | | | | S | | | | | | N | | | | G | |
| MYD4 | | | | | | | | | | | V | | | | | | |
| MYD5 | | | | | | | | | | | | | | | | | |
| MYD6 | | | | | | | | | | | | | E | | | | |
| MYD7 | | | | | | | | | | | V | | | | | | |
| MYD8 | | | | | | | | | | | | | | | R | | |
| MYD9 | | | | | | | | | | | V | | | | | | |
| MYD10 | | | | | | | | | | N | | | | | | G | |
| MYD11 | | | | | | | | | | | | | | | | G | |
| MYD12 | | | | K | | | | | Y | | V | | | | | | N |
| MYD13 | | | | | | | | | | | | | | | | | |
| MYD14 | | | | | | | | A | | | V | | | | | | |
| MYD15 | | | | | | | | | | | | | | | | | |
| MYD16 | | | | | | | | | | | | | | | | | |
| MYD17 | | | | | | | | | | | | | | | | | |
| MYD18 | | | | G | | | | | | | | | | | | G | |
| MYD19 | | | | | | | I | | | | | | | | | | |
| MYD20 | | | | | | | | | | | | | | | | G | |
| MYD21 | | M | | | | | | | | | | | | | | | |
| MYD22 | | | | | | | | | | | V | | | | | | |
| MYD23 | | | | | | | | | | | | | | | R | | |
| MYD24 | T | | G | G | | | | | | | | | | | | | |
| MYD25 | | | | | | | | | | | | | | | | | |

| Residue # | 90 | 92 | 97 | 98 | 105 | 109 | 112 | 113 | 116 | 118 | 127 | 129 | # of repeats |
|---|---|---|---|---|---|---|---|---|---|---|---|---|---|
| wt Axl | I | V | I | T | T | Q | V | F | H | T | G | E | |
| MYD1 | | A | | | | | | | | | R | | 62 |
| MYD2 | | A | | | | | | | | | E | | 21 |
| MYD3 | | A | | | | | | | | | | | 1 |
| MYD4 | | | R | | | | | | R | | | | 1 |
| MYD5 | | A | | | | | | | | | | | 16 |
| MYD6 | | | | | | | | | | | | | 1 |
| MYD7 | | | | | | | | | | | | | 7 |
| MYD8 | V | A | | | | | | | | | | | 10 |
| MYD9 | | D | | | | | | | | | | | 1 |
| MYD10 | | | | | | | | | | | | | 1 |
| MYD11 | | A | | | | | | | | | | | 2 |
| MYD12 | | A | | A | | | | | | | | | 1 |
| MYD13 | | A | | | | R | | | | | | | 1 |
| MYD14 | V | | | | M | | | | | | | K | 1 |
| MYD15 | | G | | | | | | | | | | | 2 |
| MYD16 | | A | | | | | A | L | | A | | | 1 |
| MYD17 | | A | | P | | | | | | | | | 1 |
| MYD18 | | | | | | | | | | | | | 1 |
| MYD19 | | A | | | | | | | | | | | 2 |
| MYD20 | | | | | | | | | | | | | 3 |
| MYD21 | | A | | | | | | | | | | | 1 |
| MYD22 | | | | | | | | L | | | | | 1 |
| MYD23 | | A | | | | | | | | | | | 1 |
| MYD24 | | A | | | | | | | | | | | 1 |
| MYD25 | M | A | | | | | | | | | | | 1 |

| | |
|---|---|
| TOTAL READS: | 141 |

To gain an understanding of the structural basis for this affinity improvement, MYD1-72 was crystallized in complex with Gas6. A low-resolution (3.5 Å) structure was obtained in which the overall geometry and stoichiometry of the previous structures were retained. No significant backbone changes were observed relative to the MYD1 structure (r.m.s.d. 0.25 Å), including within the loop that contains the A72V mutation and within Helix A on Gas6 (FIG. 6b). The enhancements in affinity from A72V were therefore due to further optimization of favorable contacts between Gas6 and Axl.

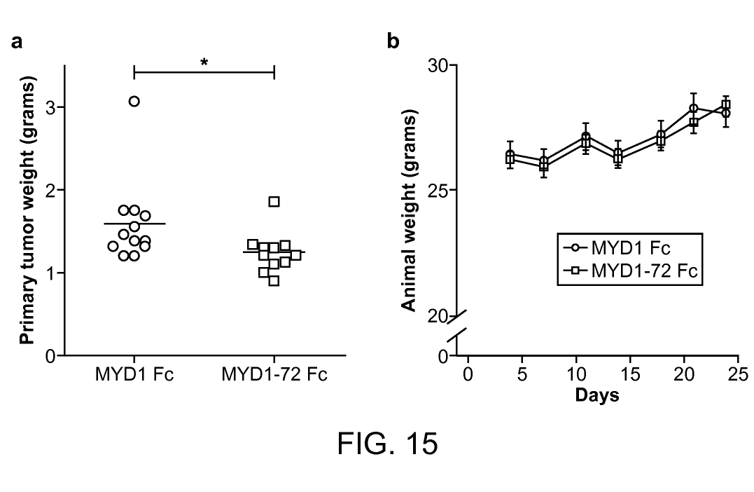

To determine whether the additional affinity enhancements afforded improved efficacy, MYD1-72 Fc and MYD1 Fc were compared in the 4T1 breast cancer model. To observe differences between the two variants, a dosing concentration of 0.5 mg/kg was selected, as this was the approximate mid-point concentration of MYD1 Fc required for sequestering serum Gas6 (FIG. 5c). A time course study following a single 0.5 mg/kg dose in non-tumor bearing mice revealed MYD1-72 Fc exhibited a greater ability to sequester Gas6 (FIG. 6c). MYD1-72 Fc also demonstrated an improved ability to inhibit lung metastases compared to MYD1 Fc (FIG. 6d and FIG. 15), which further illustrated the relationship between the affinity of the therapeutic and its efficacy.

In this study, ultra-high affinity Axl-based protein therapeutics that bind Gas6 with apparent affinities as low as 93 fM were engineered. Structural analysis of the Axl variants in complex with Gas6 revealed that the affinity enhancement was in part due to mutations in Axl causing stabilization of a helix on Gas6 through N-terminal capping. MYD1 Fc and MYD1-72 Fc were the first antagonists of the Gas6/Axl signaling axis that had affinities significantly greater than the native interaction, and were potent inhibitors of metastasis in vivo. These results further supported the critical role of Axl in disease progression while illustrating the value of Gas6 as a therapeutic target. These studies also highlighted that the therapeutic efficacy of a receptor decoy was increased by increasing its ligand binding affinity, and suggested MYD1-72 Fc as a candidate for clinical development.

### Methods Summary

Engineering and characterization of high affinity Axl variants.

The human Axl Ig1 gene was used as a template for error-prone PCR. The resulting library was displayed on yeast as previously described, and sorted by flow cytometry^{20,21}. Expression on the cell surface was detected using chicken anti-c-Myc (Invitrogen) and goat anti-chicken Alexa Fluor 555 (Invitrogen) antibodies, and a mouse anti-6×HIS Hylight Fluor 488 (Anaspec) antibody against the HIS tag on Gas6 (R&D Systems) was used to probe Gas6 binding. The binding affinities of recombinantly produced Axl Ig1 variants to Gas6 were determined using the kinetic exclusion assay (KinExA). Temperature melts were performed using a Jasco J-815 circular dichroism spectropolarimeter to determine the thermal stabilities of Axl Ig1 variants.

### Structural Determination of Gas6/Axl Complex.

Soluble Axl Ig1-2 variants were produced in E. coli, and Gas6 was produced in human embryonic kidney (HEK) cells, both as previously described^{19,25}. Crystals were obtained by the hanging drop vapor diffusion method. Data was collected at the Stanford Synchrotron Radiation Laboratory. Crystal structures were solved by molecular replacement with MOLREP²⁸, subjected to manual model building using COOT²⁹, and refined using REFMAC³⁰. All structure figures were made in PyMOL³¹.

### In Vivo Tumor Models.

All procedures involving animals and their care and use were approved by the Institutional Animal Care and Usage Committee of Stanford University in accordance with institutional guidelines. Six week old, female nude (nu/nu) mice (Jackson Laboratory) were used for all in vivo studies. 4T1 luciferase cells were injected orthotopically into the mammary fat pad, and skov3.ip, OVCAR8 and PDA-1 cells were injected intraperitoneally to establish disease models. Mice were treated twice a week by intraperitoneal or intravenous injection of 0.5-10 mg/kg of appropriate protein or control.

### Methods

### Synthesis of Yeast-Displayed Axl Library.

DNA encoding human Axl Ig1, amino acids Ala19-Pro131, was cloned into the pCT yeast display plasmid^{20,21} using NheI and BamHI restriction sites. Sequence numbering was done to match that used in Sasaki et al¹⁹ to facilitate comparisons to their work with the wild-type proteins. An error-prone library was created using the Axl Ig1 DNA as a template and mutations were introduced by using low-fidelity Taq polymerase (Invitrogen) and the nucleotide analogs 8-oxo-dGTP and dPTP (TriLink Biotech). Six separate PCR reactions were performed in which the concentration of analogs and the number of cycles were varied to obtain a range of mutation frequencies; five cycles (200 μM), ten cycles (2, 20, or 200 μM), and 20 cycles (2 or 20 μM). Products from these reactions were amplified using forward and reverse primers each with 50 by homology to the pCT plasmid in the absence of nucleotide analogs. Amplified DNA was purified using gel electrophoresis and pCT plasmid was digested with NheI and BamHI. Purified mutant cDNA and linearized plasmid were electroporated in a 5:1 ratio by weight into EBY100 yeast where they were assembled in vivo through homologous recombination^{20,21}_ENREF_21_ENREF_18. Library size was estimated to be 7.4×10⁷ by dilution plating.

### Library Screening.

Yeast displaying high affinity Axl variants were isolated from the library using fluorescence-activated cell sorting (FACS). For FACS rounds 1-3, equilibrium binding sorts were performed in which yeast were incubated at room temperature in phosphate buffered saline with 1 mg/ml BSA (PBSA) with the following concentrations of Gas6 (R&D Systems): sort 1) 10 nM Gas6 for 3 h; sort 2) 2 nM Gas6 for 3 h; sort 3) 0.2 nM Gas6 for 24 h. After incubation with Gas6, yeast were pelleted, washed, and resuspended in PBSA with 1:250 of chicken anti-c-Myc (Invitrogen) for 1 h at 4° C. Yeast were then washed, pelleted and secondary labeling was performed for on ice for 30 min using PBSA with a 1:100 dilution of goat anti-chicken Alexa Fluor 555 (Invitrogen) and mouse anti-HIS Hilyte Fluor 488 (Anaspec).

For FACS rounds 4-6, kinetic off-rate sorts were conducted in which yeast were incubated with 2 nM Gas6 for 3 hours at room temperature, after which cells were washed twice to remove excess unbound Gas6, and resuspended in PBSA containing a ˜50 fold molar excess of Axl Fc (R&D Systems) to render unbinding events irreversible. The length of the unbinding step was as follows: sort 4) 4 h; sort 5) 4 h; sort 6) 24 h, with all unbinding reactions performed at room temperature. During the last hour of the dissociation reaction, chicken anti-c-Myc was added to a final dilution of 1:250. Yeast were pelleted, washed, and secondary labeling was performed as previously described. Labeled yeast were sorted by FACS using a Vantage SE flow cytometer (Stanford FACS Core Facility) and CellQuest software (Becton Dickinson). Sorts were conducted such that the 1-3% of clones with the highest Gas6 binding/c-Myc expression ratio were selected, enriching the library for clones with the highest binding affinity to Gas6. In sort 1, 10⁸ cells were screened and subsequent rounds analyzed a minimum of ten-fold the number of clones collected in the prior sort round to ensure adequate sampling of the library diversity. Selected clones were propagated and subjected to further rounds of FACS. Following sorts 5 and 6, plasmids were recovered using a Zymoprep kit (Zymo Research Corp.), transformed into XL-1 blue supercompetent cells, and isolated using plasmid miniprep kit (Qiagen). Sequencing was performed by Sequetech Corp.

Analysis of yeast-displayed sort products and individual Axl variants was performed using the same reagents and protocols and described for the library sorts. Samples were analyzed on a FACSCalibur (BD Biosciences) and data was analyzed using FlowJo software (Treestar Inc.).

### Recombinant Protein Production.

Axl Ig1 variants were cloned into the pPIC9K plasmid (Invitrogen) with N- and C-terminal FLAG and 6×HIS tags, respectively. Plasmid DNA was transformed into the yeast P. pastoris GS115 strain according to the manufacturer's protocol and proteins were purified from culture supernatant using nickel affinity chromatography. N-linked glycosylation was removed using endoglycosidase H (EndoHf, New England Biolabs) and monomeric Axl Ig1 was further purified using size exclusion chromatography. The Gas6 and Axl constructs used for crystallography studies were produced as previously described^{19,25}. Axl Fc fusions were obtained by cloning the cDNA encoding the signal peptide and extracellular domain (amino acids 1-440) of Axl into the cytomegalovirus-driven pAdd2 adenoviral shuttle vector directly upstream of the gene encoding a human IgG, Fc domain. Human embryonic kidney (HEK) cells were transiently transfected with pAdd2 plasmid DNA using the FreeStyle Max 293 Expression System (Invitrogen). Axl Fc fusions were purified from culture supernatant using protein A affinity chromatography and disulfide-linked dimers were isolated by size exclusion chromatography.

### Kinetic Exclusion Assay (KinExA).

A KinExA 3200 instrument (Sapidyne Instruments Inc.) was used to measure equilibrium binding and association rate constants for the Gas6-Axl interactions. For all experiments, MYD1 Fc coated polymethyl methacrylate (PMMA) beads were used as a capture reagent. Adsorption coating of 200 mg of beads was performed using 45 μg of protein at room temp for 1 h. The protein solution was then removed and beads were blocked using PBS with 1% BSA for 2 h at room temp. Beads were stored in blocking buffer at 4° C. and used within two weeks. All binding reactions were carried out in phosphate buffered saline with 1 mg/ml BSA and 0.02% sodium azide.

For equilibrium binding studies, three independent titrations were completed for each Axl Ig1 or Axl Fc fusion unless otherwise noted. Depending on the affinity of interaction, three of the following four titrations were performed: 1) 1 ml reactions of 5 nM Gas6, Axl serially diluted 1:2 twelve times starting at 30 nM, 3 h RT incubation; 2) 2 ml reactions of 500 pM Gas6, Axl serially diluted 1:2.5 twelve times starting at 10 nM, 18 h RT incubation; 3) 12 ml reactions of 50 pM Gas6, Axl serially diluted 1:3 twelve times starting at 9 nM, 1 day RT incubation; 4) 12 ml reactions of 15 pM Gas6, Axl serially diluted 1:3 twelve times starting at 1 nM, 4 day RT incubation. After the appropriate incubation time, reactions were flowed over MYD1 Fc coated beads and captured free Gas6 was measured using an anti-6×HIS Dylight 649 antibody (Rockland Immunochemicals Inc.). Each sample was analyzed twice and data from the three independent equilibrium binding experiments were globally analyzed using n-curve analysis in the KinExA Pro 3.6.2 software (Sapidyne Instruments Inc.) to obtain the K_{d} value.

To analyze the association rate of the interactions, the direct inject method was used. For these experiments, 1 μM Axl was serially diluted 1:2.5, and equal volumes of each Axl sample and 500 nM Gas6 were briefly mixed and flowed over the capture beads. Free Gas6 was detected as described and the data was fit using the KinExA Pro 3.6.2 software to obtain the association rate (kW of the interaction. The dissociation rate (k_{off}) was calculated as the product of the K_{d} and the kₒₙ.

To test binding of Protein S (OriGene) to MYD1, 700 μl of a 10 nM solution was flowed over the capture beads. Binding of Protein S to the beads was probed using an anti-c-Myc Dylight 649 antibody (Rockland Immunochemicals Inc.).

### Circular Dichroism Spectroscopy.

To measure the thermal stabilities of the Axl Ig1 variants, temperature melts were performed using a Jasco J-815 circular dichroism spectropolarimeter. Recombinant protein was diluted to 10 μM in PBS and ellipticity was monitored at 206 nm as temperature was increased from 20-80° C. at a rate of 1° C./min. The resulting temperature melt was fit to a two-state unfolding curve in order to calculate the Tₘ.

### Crystallization and Data Collection for Gas6/Axl Co-Complexes.

Identical conditions were used for both Gas6/MYD1 and Gas6/MYD1-72 co-complexes. Purified wild-type Gas6 and Axl Ig1-2 variants were mixed in a 1:1 molar ratio and allowed to complex at room temperature for 24 h. The co-complexes were purified using size exclusion chromatography to remove any uncomplexed components, and were buffer exchanged into 25 mM Na-HEPES, 150 mM NaCl, and 1 mM calcium acetate to a final concentration of 10 mg/ml. Crystals for the Gas6/MYD1 co-complex were grown at room temperature by the hanging-drop vapor-diffusion method with a 1:1 mixture (1.2 μl each) of the complex solution (6.2 mg/ml) and the well solution containing 0.4M Li₂SO₄, 0.1M Tris-HCl (pH 8.5), 5% glycerol, and 2 mM Ni₂SO₄. The Gas6/MYD1-72 co-complex crystallized under similar conditions by using 5.6 mg/mL protein and 0.15M Li₂SO₄ as the precipitant. For cryocooling, the crystals were dipped in a solution containing 38 parts of 1M Li₂SO₄, 0.1M Tris-HCl (pH 8.5), 0.1M NaCl and 12 parts of 100% glycerol. Diffraction data sets for the Gas6/MYD1 co-complex (3.1 Å) and the Gas6/MYD1-72 co-complex (3.5 Å) were collected at 100 K using Stanford Synchrotron Radiation Lightsource (SSRL) beamlines 7-1 and 12-2, respectively. Data were indexed and integrated using the XDS package³². The crystals belong to space group P3₂21 and contain two monomers per symmetric unit. The crystallographic data are summarized in Table 3.

### Structure Determination and Refinement.

Initial phases were obtained by molecular replacement by using the program MOLREP²⁸ and the coordinates of the wild type Gas6/Axl Ig1-2 crystal structure (PDB ID: 2C5D) as the search model. Density corresponding to the loop on Axl from residue 541 to 550 was visible for chain A. This loop is missing in the wild-type structure. After several cycles of manual model building using COOT²⁹ and refinement using REFMAC³⁰, the final R_{(working)} and Rₜᵣₑₑ values were 20.2% and 24.3%, respectively. The structure of the Gas6/MYD1-72 co-complex was solved by molecular replacement, using the coordinates of the Gas6/MYD1 co-complex as the search model. Manual model building and refinement using COOT and REFMAC resulted in final R_{(working)} and Rₜᵣₑₑ values of 20.3% and 24.5%, respectively. The refinement statistics are provided in Table 3.

### Analysis of Intermolecular Contacts.

The binding interface in the Gas6/MYD1 co-complex was analyzed using the PDBePISA³³ server v1.47 [21/3/2013] (located on the World Wide Web at ebi.ac.uk/pdbe/prot_int/pistart.html) utilizing a cutoff of 3.5 Å and 4.0 Å for hydrogen bonds and electrostatic interactions, respectively. The results are shown in Table 4. Normalized B-factors shown in FIG. 4f were calculated as described previously³⁴. For this analysis the wild-type Gas6/Axl complex (PDB ID: 2C5D) was refined in a manner identical to the Gas6/MYD1 complex to facilitate comparison.

### Analysis of In Vivo Gas6 Sequestration by Axl Fc Fusions.

For the dose response studies, six week old, female nude (nu/nu) mice (Jackson Laboratory) were administered a single dose of MYD1 Fc ranging from 0.1-1 mg/kg via tail vein injection. All doses were formulated in a 200 μl volume. Two mice were analyzed per condition and untreated mice were used to determine normal Gas6 levels. Twelve hours post-injection, retro-orbital bleeds were performed to obtain blood samples from which serum was isolated. The amount of free Gas6 in each sample was determined using a sandwich ELISA. In this assay, MYD1 Fc was used as a capture reagent in order to ensure the detection of free, unbound Gas6, and not Gas6/Axl Fc complexes. Detection of Gas6 was carried out using a biotinylated polyclonal anti-mouse Gas6 antibody (R&D Systems) and streptavidin HRP (Trevigen Inc.).

For time course studies, the protocols were performed as described above except that all mice received a single dose (0.5 or 1 mg/kg) of Axl Fc. Blood collection was done at 2, 12, 24, 36 or 48 h post-administration and free Gas6 was measured.

### In Vivo Tumor Models.

For all studies, six week old, female nude (nu/nu) mice (Jackson Laboratory) were used. All procedures involving animals and their care and use were approved by the Institutional Animal Care and Usage Committee of Stanford University.

To establish orthotopic mammary tumors, 5×10⁴ 4 T1 luciferase cells³⁵ (kindly provided by Dr. Marta Vilalta, Stanford University) were injected in 50 μl of DMEM into the 4^{th} inguinal mammary fat pad. Tumor establishment was confirmed using an IVIS 200 imager (PerkinElmer Inc.) 3 days post-tumor inoculation. For both studies, treatment began 4 days post-inoculation and consisted of a 0.5, 1, or 10 mg/kg dose of Axl Fc administered intravenously (IV) via tail vein injection in a 200 μl volume. Treatment was administered twice a week for 3 weeks, for a total of 6 doses. Animal weight and primary tumor growth were measured over the course of the study, with tumor volume being calculated using the formula (π/6)*l*w*h. On day 24, mice were sacrificed 10 minutes after receiving a single intraperitoneal (IP) injection of D-luciferin. Ex-vivo bioluminescent imaging of the lungs and spleen was performed using an IVIS 200 to quantify lung metastases. Further analysis of lung metastatic burden was performed by detecting luciferase signal using TaqMan® quantitative-PCR. Briefly, genomic DNA was isolated from whole mouse lung and assayed to detect tumor specific luciferase expression and the expression of vimentin was examined as a house-keeping gene. By comparing the Ct values of vimentin and luciferase (ΔCT), a score for relative tumor burden was calculated using the formula (10,000×1/2^{corrΔcT}). CorrΔCT is a ΔCT value that includes correction for the difference in luciferase copy number in each tumor line. Multiplex PCR reactions consisted of 50 nM vimentin forward and reverse primers (forward 5′-AGCTGCTAACTACCAGGACACTATTG-3′, reverse 5′-CGAAGGTGACGAGCCATCTC-3′), 50 nM vimentin probe (5′-CCTTCATGTTTTGGATCTCATCCTGCAGG-3′) and both luciferase primers and probe were designed and purchased as Primetime® Assay kit (Integrated DNA Technologies Inc.). At the conclusion of the study, primary tumors were excised, weighted and fixed. Immunohistochemical staining of the proliferation marker Ki67 was performed to evaluate tumor proliferation status. Briefly, tumor sections were subjected to antigen retrieval using 10 mM citric acid and were probed with primary anti-Ki67 antibody (Abcam) overnight at 4° C. followed by secondary detection using streptavidin-HRP.

To generate diffuse metastatic disease in the human ovarian and pancreatic cancer models, 1×10⁶ skov3.ip cells' (kindly provided by Dr. Gordon Mills, M.D. Anderson Cancer Center), 5×10⁶ OVCAR8 cells⁷ (NCI Frederick DCTD tumor cell line repository), or 4×10⁴ PDA-1 cells³⁶_ENREF_36 (kindly provided by Dr. Edgar Engleman, Stanford University) were injected IP and allowed to establish for one week, two weeks, or 4 days, respectively, prior to dosing. Axl Fc fusions were administered at 1 or 10 mg/kg twice a week via IP injections for three weeks. For the ovarian cancer models, animals were sacrificed on day 28 (skov3.ip) or day 35 (OVCAR8), and metastatic burden was assessed by counting the number of visible metastatic lesions in the peritoneal cavity as well as excising and weighting all tumor tissue. The overall weight of all tumor tissue was measured in the PDA-1 model on day 24.

### Toxicology Study of MYD1 Fc.

Six week old non-diseased, female nude (nu/nu) mice (Jackson Laboratory) were injected IV via tail vein with a 10 mg/kg dose of MYD1 Fc twice a week for four weeks. Animal weight was measured over the course of the study. After 28 days, mice were sacrificed and complete blood counts and chemistry panels were performed (Stanford Veterinary Service Center).

### Statistical Analysis.

Differences between groups in all in vivo experiments were examined for statistical significance using a two-tailed Student's t-test with the exception of primary tumor volumes for which a one-way analysis of variance (ANOVA) was performed. A P value <0.05 was considered significant.

### REFERENCES

1 Verma, A., Warner, S. L., Vankayalapati, H., Bearss, D. J. & Sharma, S. Targeting Axl and Mer Kinases in Cancer. Mol Cancer Ther 10, 1763-1773, (2011).
2 Vajkoczy, P. et al. Dominant-negative inhibition of the Axl receptor tyrosine kinase suppresses brain tumor cell growth and invasion and prolongs survival. Proc Natl Acad Sci USA 103, 5799-5804, (2006).
3 Duijkers, F. A., Meijerink, J. P., Pieters, R. & van Noesel, M. M. Downregulation of Axl in non-MYCN amplified neuroblastoma cell lines reduces migration. Gene 521, 62-68, (2013).
4 Ou, W. B. et al. AXL regulates mesothelioma proliferation and invasiveness. Oncogene 30, 1643-1652, (2011).
5 Asiedu, M. K. et al. AXL induces epithelial-to-mesenchymal transition and regulates the function of breast cancer stem cells. Oncogene, (2013).
6 Gjerdrum, C. et al. Axl is an essential epithelial-to-mesenchymal transition-induced regulator of breast cancer metastasis and patient survival. Proc Natl Acad Sci USA 107, 1124-1129, (2010).
7 Rankin, E. B. et al. AXL Is an Essential Factor and Therapeutic Target for Metastatic Ovarian Cancer. Cancer Res 70, 7570-7579, (2010).
8 Zhang, Z. et al. Activation of the AXL kinase causes resistance to EGFR-targeted therapy in lung cancer. Nat Genet 44, 852-860, (2012).
9 Ye, X. et al. An anti-Axl monoclonal antibody attenuates xenograft tumor growth and enhances the effect of multiple anticancer therapies. Oncogene 29, 5254-5264, (2010).
10 Li, Y. et al. Axl as a potential therapeutic target in cancer: role of Axl in tumor growth, metastasis and angiogenesis. Oncogene 28, 3442-3455, (2009).
11 Cerchia, L. et al. Targeting Axl With an High-affinity Inhibitory Aptamer. Mol Ther 20, 2291-2303, (2012).
12 Holland, S. J. et al. R428, a selective small molecule inhibitor of Axl kinase, blocks tumor spread and prolongs survival in models of metastatic breast cancer. Cancer Res 70, 1544-1554, (2010).
13 Park, I. K. et al. Inhibition of the receptor tyrosine kinase Axl impedes activation of the FLT3 internal tandem duplication in human acute myeloid leukemia: implications for Axl as a potential therapeutic target. Blood 121, 2064-2073, (2013).
14 Kariolis, M. S., Kapur, S. & Cochran, J. R. Beyond antibodies: using biological principles to guide the development of next-generation protein therapeutics. Curr Opin Biotechnol, (2013).
15 Varnum, B. C. et al. Axl receptor tyrosine kinase stimulated by the vitamin K-dependent protein encoded by growth-arrest-specific gene 6. Nature 373, 623-626, (1995).
16 Stitt, T. N. et al. The Anticoagulation Factor Protein-S and Its Relative, Gas6, Are Ligands for the Tyro 3/Axl Family of Receptor Tyrosine Kinases. Cell 80, 661-670, (1995).
17 Rogers, A. E. et al. Mer receptor tyrosine kinase inhibition impedes glioblastoma multiforme migration and alters cellular morphology. Oncogene 31, 4171-4181, (2012).
18 Schlegel, J. et al. MERTK receptor tyrosine kinase is a therapeutic target in melanoma. J Clin Invest, (2013).
19 Sasaki, T. et al. Structural basis for Gas6-Axl signalling. Embo J 25, 80-87, (2006).
20 Boder, E. T. & Wittrup, K. D. Yeast surface display for screening combinatorial polypeptide libraries. Nat Biotechnol 15, 553-557, (1997).
21 Chao, G. et al. Isolating and engineering human antibodies using yeast surface display (vol 1, pg 755, 2006). Nat Protoc 1, (2006).
22 Darling, R. J. & Brault, P. A. Kinetic exclusion assay technology: Characterization of molecular interactions. Assay Drug Dev Techn 2, 647-657, (2004).
23 Hafizi, S. & Dahlback, B. Gas6 and protein S. Vitamin K-dependent ligands for the Axl receptor tyrosine kinase subfamily. FEBS J 273, 5231-5244, (2006).
24 Manfioletti, G., Brancolini, C., Avanzi, G. & Schneider, C. The protein encoded by a growth arrest-specific gene (gash) is a new member of the vitamin K-dependent proteins related to protein S, a negative coregulator in the blood coagulation cascade. Mol Cell Biol 13, 4976-4985, (1993).
25 Sasaki, T. et al. Crystal structure of a C-terminal fragment of growth arrest-specific protein Gas6-Receptor tyrosine kinase activation by laminin G-like domains. J Biol Chem 277, 44164-44170, (2002).
26 Aurora, R. & Rose, G. D. Helix capping. Protein Sci 7, 21-38, (1998).
27 Naora, H. & Montell, D. J. Ovarian cancer metastasis: integrating insights from disparate model organisms. Nat Rev Cancer 5, 355-366, (2005).
28 Vagin, A. & Teplyakov, A. Molecular replacement with MOLREP. Acta Crystallogr D Biol Crystallogr 66, 22-25, (2010).
29 Emsley, P. & Cowtan, K. Coot: model-building tools for molecular graphics. Acta Crystallogr D 60, 2126-2132, (2004).
30 Murshudov, G. N., Vagin, A. A. & Dodson, E. J. Refinement of macromolecular structures by the maximum-likelihood method. Acta Crystallogr D Biol Crystallogr 53, 240-255, (1997).
31 Schrodinger, LLC. The PyMOL Molecular Graphics System, Version 1.3r1 (2010).
32 Kabsch, W. Xds. Acta Crystallogr D Biol Crystallogr 66, 125-132, (2010).
33 Krissinel, E. & Henrick, K. Inference of macromolecular assemblies from crystalline state. J Mol Biol 372, 774-797, (2007).
34 Yuan, Z., Zhao, J. & Wang, Z. X. Flexibility analysis of enzyme active sites by crystallographic temperature factors. Protein Eng 16, 109-114, (2003).
35 Aslakson, C. J. & Miller, F. R. Selective events in the metastatic process defined by analysis of the sequential dissemination of subpopulations of a mouse mammary tumor. Cancer Res 52, 1399-1405, (1992).
36 Tseng, W. W. et al. Development of an orthotopic model of invasive pancreatic cancer in an immunocompetent murine host. Clin Cancer Res 16, 3684-3695, (2010).

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

### Listing of Sequences:

SEQ ID NO: 1 is the amino acid sequence of human
wild type AXL
MAWRCPRMGRVPLAWCLALCGWACMAPRGTQAEESPFVGNPGNITGARGL
TGTLRCQLQVQGEPPEVHWLRDGQILELADSTQTQVPLGEDEQDDWIVVS
QLRITSLQLSDTGQYQCLVFLGHQTFVSQPGYVGLEGLPYFLEEPEDRTV
AANTPFNLSCQAQGPPEPVDLLWLQDAVPLATAPGHGPQRSLHVPGLNKT
SSFSCEAHNAKGVTTSRTATITVLPQQPRNLHLVSRQPTELEVAWTPGLS
GIYPLTHCTLQAVLSNDGMGIQAGEPDPPEEPLTSQASVPPHQLRLGSLH
PHTPYHIRVACTSSQGPSSWTHWLPVETPEGVPLGPPENISATRNGSQAF
VHWQEPRAPLQGTLLGYRLAYQGQDTPEVLMDIGLRQEVTLELQGDGSVS
NLTVCVAAYTAAGDGPWSLPVPLEAWRPGQAQPVHQLVKEPSTPAFSWPW
WYVLLGAVVAAACVLILALFLVHRRKKETRYGEVFEPTVERGELVVRYRV
RKSYSRRTTEATLNSLGISEELKEKLRDVMVDRHKVALGKTLGEGEFGAV
MEGQLNQDDSILKVAVKTMKIAICTRSELEDFLSEAVCMKEFDHPNVMRL
IGVCFQGSERESFPAPVVILPFMKHGDLHSFLLYSRLGDQPVYLPTQMLV
KFMADIASGMEYLSTKRFIHRDLAARNCMLNENMSVCVADFGLSKKIYNG
DYYRQGRIAKMPVKWIAIESLADRVYTSKSDVWSFGVTMWEIATRGQTPY
PGVENSEIYDYLRQGNRLKQPADCLDGLYALMSRCWELNPQDRPSFTELR
EDLENTLKALPPAQEPDEILYVNMDEGGGYPEPPGAAGGADPPTQPDPKD
SCSCLTAAEVHPAGRYVLCPSTTPSPAQPADRGSPAAPGQEDGA
SEQ ID NO: 2 is the amino acid sequence of human
wild type MER
MGPAPLPLLLGLFLPALWRRAITEAREEAKPYPLFPGPFPGSLQTDHTPL
LSLPHASGYQPALMFSPTQPGRPHTGNVAIPQVTSVESKPLPPLAFKHTV
GHIILSEHKGVKFNCSISVPNIYQDTTISWWKDGKELLGAHHAITQFYPD
DEVTAIIASFSITSVQRSDNGSYICKMKINNEEIVSDPIYIEVQGLPHFT
KQPESMNVTRNTAFNLTCQAVGPPEPVNIFWVQNSSRVNEQPEKSPSVLT
VPGLTEMAVFSCEAHNDKGLTVSKGVQINIKAIPSPPTEVSIRNSTAHSI
LISWVPGFDGYSPFRNCSIQVKEADPLSNGSVMIFNTSALPHLYQIKQLQ
ALANYSIGVSCMNEIGWSAVSPWILASTTEGAPSVAPLNVTVFLNESSDN
VDIRWMKPPTKQQDGELVGYRISHVWQSAGISKELLEEVGQNGSRARISV
QVHNATCTVRIAAVTRGGVGPFSDPVKIFIPAHGWVDYAPSSTPAPGNAD
PVLIIFGCFCGFILIGLILYISLAIRKRVQETKFGNAFTEEDSELVVNYI
AKKSFCRRAIELTLHSLGVSEELQNKLEDVVIDRNLLILGKILGEGEFGS
VMEGNLKQEDGTSLKVAVKTMKLDNSSQREIEEFLSEAACMKDFSHPNVI
RLLGVCIEMSSQGIPKPMVILPFMKYGDLHTYLLYSRLETGPKHIPLQTL
LKFMVDIALGMEYLSNRNFLHRDLAARNCMLRDDMTVCVADFGLSKKIYS
GDYYRQGRIAKMPVKWIAIESLADRVYTSKSDVWAFGVTMWEIATRGMTP
YPGVQNHEMYDYLLHGHRLKQPEDCLDELYEIMYSCWRTDPLDRPTFSVL
RLQLEKLLESLPDVRNQADVIYVNTQLLESSEGLAQGSTLAPLDLNIDPD
SIIASCTPRAAISVVTAEVHDSKPHEGRYILNGGSEEWEDLTSAPSAAVT
AEKNSVLPGERLVRNGVSWSHSSMLPLGSSLPDELLFADDSSEGSEVLM
SEQ ID NO: 3 is the amino acid sequence of human
wild type Tyro3
MALRRSMGRPGLPPLPLPPPPRLGLLLAALASLLLPESAAAGLKLMGAPV
KLTVSQGQPVKLNCSVEGMEEPDIQWVKDGAVVQNLDQLYIPVSEQHWIG
FLSLKSVERSDAGRYWCQVEDGGETEISQPVWLTVEGVPFFTVEPKDLAV
PPNAPFQLSCEAVGPPEPVTIVWWRGTTKIGGPAPSPSVLNVTGVTQSTM
FSCEAHNLKGLASSRTATVHLQALPAAPFNITVTKLSSSNASVAWMPGAD
GRALLQSCTVQVTQAPGGWEVLAVVVPVPPFTCLLRDLVPATNYSLRVRC
ANALGPSPYADWVPFQTKGLAPASAPQNLHAIRTDSGLILEWEEVIPEAP
LEGPLGPYKLSWVQDNGTQDELTVEGTRANLTGWDPQKDLIVRVCVSNAV
GCGPWSQPLVVSSHDRAGQQGPPHSRTSWVPVVLGVLTALVTAAALALIL
LRKRRKETRFGQAFDSVMARGEPAVHFRAARSFNRERPERIEATLDSLGI
SDELKEKLEDVLIPEQQFTLGRMLGKGEFGSVREAQLKQEDGSFVKVAVK
MLKADIIASSDIEEFLREAACMKEFDHPHVAKLVGVSLRSRAKGRLPIPM
VILPFMKHGDLHAFLLASRIGENPFNLPLQTLIRFMVDIACGMEYLSSRN
FIHRDLAARNCMLAEDMTVCVADFGLSRKIYSGDYYRQGCASKLPVKWLA
LESLADNLYTVQSDVWAFGVTMWEIMTRGQTPYAGIENAEIYNYLIGGNR
LKQPPECMEDVYDLMYQCWSADPKQRPSFTCLRMELENILGQLSVLSASQ
DPLYINIERAEEPTAGGSLELPGRDQPYSGAGDGSGMGAVGGTPSDCRYI
LTPGGLAEQPGQAEHQPESPLNETQRLLLLQQGLLPHSSC
SEQ ID NO: 4 is the amino acid sequence region
L295-T317 of human GAS6
LRMFSGTPVIRLRFKRLQPT
SEQ ID NO: 5 is the amino acid sequence region
E356-P372 of human GAS6
EIVGRVTSSGP
SEQ ID NO: 6 is the amino acid sequence region
R389-N396 of human GAS6
RNLVIKVN
SEQ ID NO: 7 is the amino acid sequence region
D398-A406 of human GAS6
DAVMKIAVA
SEQ ID NO: 8 is the amino acid sequence region
E413-H429 of human GAS6
ERGLYHLNLTVGIPFH
SEQ ID NO: 9 is the amino acid sequence region
W450-M468 of human GAS6
WLNGEDTTIQETVVNRM
SEQ ID NO: 10 is the (GLY)₄SER linker sequence
GGGGS

1. A method of measuring the amount of Gas6 in a sample, the method comprising:
(a) contacting a GAS6 capture reagent with a sample to produce a contacted sample, wherein the capture reagent is capable of binding to GAS6 polypeptide with increased affinity compared to wild-type AXL; and
(b) measuring the amount of GAS6 polypeptide that is bound to the capture reagent.

2. The method according to claim 1, wherein the capture reagent is capable of binding to the major and minor AXL binding sites on a single GAS6.

3. The method according to claim 1, wherein the capture reagent is capable of inducing a conformational change of GAS6 that stabilizes of Helix A of GAS6.

4. The method according to claim 3, wherein the capture reagent is capable of causing a structural change of Helix A of GAS6 that stabilizes the bound conformation of the GAS6 through N-terminal capping of Helix A of GAS6.

5. The method according to claim 1, wherein the capture reagent is a variant AXL polypeptide comprising at least one amino acid modification relative to the wild-type AXL (SEQ ID NO: 1).

6.-8. (canceled)

9. The method according to claim 5, wherein the variant AXL polypeptide has a set of amino acid substitution(s) of the wild-type AXL sequence (SEQ ID NO. 1) selected from the group consisting of 1) Gly32Ser, Asp87Gly, Val92Ala, and Gly127Arg, 2) Glu26Gly, Val79Met, Val92Ala, and Gly127Glu, 3) Asn33Ser, Ser74Asn, Asp87Gly, and Val92Ala, 4) Ala72Val, Ile97Arg, and His116Arg, 5) Gln78Glu, 6) Ala72Val, 7) Gln86Arg, Ile90Val, and Val92Ala, 8) Ala72Val, and Val92Asp, 9) Asp65Asn, and Asp87Gly, 10) Asp87Gly, and Val92Ala, 11) Glu27Lys, His61Tyr, Ala72Val, Asp88Asn, Val92Ala, and Thr98Ala, 12) Val92Ala, Gln109Arg, 13) Thr44Ala, Ala72Val, Ile90Val, Thr105Met, and Glu129Lys, 14) Val92Gly, 15) Val92Ala, Val112Ala, Phe113Leu, and Thr118Ala, 16) Val92Ala, and Thr98Pro, 17) Glu27Gly, and Asp87Gly, 18) Thr38Ile, and Val92Ala, 19) Asp87Gly, 20) Thr23Met, and Val92Ala, 21) Ala72Val, and Phe113Leu, 22) Gln86Arg, Val92Ala, 23) Ala19Thr, Glu26Gly, Glu27Gly, and Val92Ala, 24) Ile90Met and Val92Ala, 25) Gly32Ser, and Asp87Gly, 26) Gly32Ser, and Val92Ala, 27) Gly32Ser, and Gly127Arg, 28) Asp87Gly, and Gly127Arg, 29) Val92Ala, and Gly127Arg, 30) Asp87Gly, Val92Ala, and Gly127Arg, 31) Gly32Ser, Val92Ala, and Gly127Arg, 32) Gly32Ser, Asp87Gly, and Gly127Arg, 33) Gly32Ser, Asp87Gly, and Val92Ala and 34) Gly32Ser, Ala72Val, Asp87Gly, Val92Ala, and Gly127Arg.

10-12. (canceled)

13. The method according to claim 5, wherein the variant AXL variant polypeptide comprises at least one amino acid modification selected from the group consisting of 1) A19T, 2) T23M, 3) E26G, 4) E27G or E27K 5) G32S, 6) N33S, 7) T38I, 8) T44A, 9) H61Y, 10) D65N, 11) A72V, 12) S74N, 13) Q78E, 14) V79M, 15) Q86R, 16) D87G, 17) D88N, 18) I90M or I90V, 19) V92A, V92G or V92D, 20) I97R, 21) T98A or T98P, 22) T105M, 23) Q109R, 24) V112A, 25) F113L, 26) H116R, 27) T118A, 28) G127R or G127E, and 29) G129E and a combination thereof.

14. The method according to claim 1, wherein the capture reagent is immobilized.

15. The method according to claim 14, wherein the step of measuring comprises contacting the contacted sample with a detectable GAS6 binding agent.

16. The method according to claim 15, wherein the detectable GAS6 binding agent is an anti-GAS6 antibody.

17. The method according to claim 16, wherein the method is an enzyme-linked immunosorbent assay (ELISA).

18. The method according to claim 1, wherein the sample is a biological sample.

19-20. (canceled)

21. The method according to claim 18, wherein the sample is obtained from an individual that has cancer or is suspected of having cancer.

22. The method according to, wherein the individual is undergoing treatment comprising the administration of a GAS6 inhibitor claim 21 agent to the individual.

23. The method according to claim 21, wherein a prognosis and/or diagnosis is determined for the human based on the measured amount of GAS6

24. The method according to claim 21, wherein a prognosis or diagnosis is determined for the human based on the measured amount of GAS6 relative to:
(i) a known reference standard; and/or
(ii) the amount of GAS6 measured from a reference sample.

25. A method of determining the efficacy of a GAS6 inhibitor agent, the method comprising:
measuring the amount of GAS6 in a biological sample obtained from an individual undergoing treatment, wherein:
(i) the treatment comprises the administration of a GAS6 inhibitor agent to the individual, and
(ii) the measuring is performed according to the method of claim 1.

26. The method of claim 25, further comprising, prior to the step of measuring, administering to the individual a GAS6 inhibitor agent that binds to GAS6 with higher affinity than wild type AXL (SEQ ID NO: 1).

27. The method according to claim 25, further comprising: determining a course of treatment based on the amount of GAS6 measured in the biological sample.

28. (canceled)

29. A GAS6 inhibitor agent that is capable of inducing a conformational change of GAS6 that stabilizes Helix A of GAS6, wherein the GAS6 inhibitor agent is capable of causing a structural change of Helix A of GAS6 that stabilizes the bound conformation of the GAS6 through N-terminal capping of Helix A of GAS6, and wherein the GAS6 inhibitor agent is not an AXL variant polypeptide comprising a Val92Ala mutation relative to wild type AXL (SEQ ID NO: 1).

30-33. (canceled)
